(19) United States
(12) Patent Application Publication
(10) Pub. No.: US 2025/0017881 A1
(43) Pub. Date: Jan. 16, 2025

(54) Fatty acids for ketosis control

(71) Applicant: N.V. Nutricia, Zoetermeer (NL)

(72) Inventors: Jose Pablo Silva, Utrecht (NL); Hester Meeusen, Utrecht (NL); Lotte Hendrika Johanna Dopheide, Utrecht (NL); Sebastian Tims, Utrecht (NL)

(73) Assignee: N.V. Nutricia, Zoetermeer (NL)

(21) Appl. No.: 18/815,872

(22) Filed: Aug. 27, 2024

Related U.S. Application Data
(63) Continuation of application No. PCT/EP2023/055206, filed on Mar. 1, 2023.

(30) Foreign Application Priority Data
Mar. 2, 2022 (EP) 22159658.8

(51) Int. Cl. A61K 31/19; A61K 31/194; A61K 31/455; A61K 31/706; A61K 47/36; A61K 47/38; A61P 3/00

(52) U.S. Cl. CPC A61K 31/19; A61K 31/194; A61K 31/455; A61K 31/706; A61K 47/36; A61K 47/38; A61P 3/00

(57) ABSTRACT

The invention relates to a first composition providing at least one of acetic acid or butyric acid for controlling ketosis associated with a ketogenic diet in a subject adhering to a ketogenic diet, wherein controlling ketosis involves a) administering the first composition, b) optionally administering a second composition comprising propionic acid to reduce ketone production/administering a second composition comprising propionic acid provided that blood ketone levels are above 3 mmol/L.

### FIELD OF THE INVENTION

The invention relates to the use of a first or a second composition to control or modulate levels of ketone bodies in the blood, and to combinations of a first and a second compositions to be used to that end.

### BACKGROUND

Ketosis is the state of elevated blood ketone levels resulting from ketogenic diets, caloric restriction, (therapeutic) fasting and/or supplementation with ketogenic precursors. Ketone bodies represent energy substrates for both peripheral tissues and the central nervous system. The two most abundant and physiologically significant ketone bodies are acetoacetate and p-hydroxybutyrate, while the third ketone body, acetone, is produced as a by-product that the lungs breathe off.

The metabolism of ketone bodies is associated with therapeutic effects in a wide range of therapeutic areas including neurological diseases such as epilepsy and ageing related conditions such as Alzheimer's disease, neuromuscular diseases such as amyotrophic lateral sclerosis, stroke and neurotrauma and metabolic conditions such as obesity, insulin resistance, type-2 diabetes mellitus, Glucose transporter type 1 (Glut1) deficiency, Pyruvate dehydrogenase (PDH) deficiency, glycogen storage diseases and mitochondrial diseases.

Though a ketogenic diet is effective at raising blood ketone levels and has broad therapeutic applications, patient compliance is low due to the restrictive nature of the diet. In addition to a ketogenic diet nutritional ketosis may be established or supported with exogenous ketone bodies such as an exogenous source of p-hydroxybutyrate. However, current compositions and/or dosage forms thereof lead to variable blood ketone concentrations overtime with temporarily unnecessarily high or ineffective levels and/or lack satisfying sensory properties. This not only reduces the efficacy of ketone bodies but may also increase adverse side-effects like gastrointestinal upset (Clarke et al., Regul Toxicol Pharmacol. 2012 August; 63(3):401-8; WO2018115158A1), and therewith reduce compliance of patients and/or limit their broader use. Alternative compositions are thus still needed that allow to maintain effective and at the same time well-tolerated ketone body blood concentration.

Accordingly, there is a need for compositions which can be used to support a sustained or controlled release dosage to a subject. In particular such compositions should be able to maintain a ketone body blood concentration at a therapeutically effective level, preferably with good tolerance and sensory properties. The above technical problem is solved by the embodiments as defined in the claims.

### SUMMARY OF THE INVENTION

The inventors have observed that after administration of a first composition providing at least one of butyric acid or caproic acid and in addition optionally acetic acid, the level of ketone bodies produced from a ketogenic diet is enhanced and that after administration of a second composition comprising propionic acid ketone body production is decreased. The inventors have found that the use of the first and the second composition provides a strategy to control and/or modulate blood ketone levels and obtain or maintain a state of therapeutic ketosis. It was found by the inventors that a second composition comprising propionic acid dose-dependently allows to reduce ketone production.

The inventors found that a first composition providing at least one of butyric acid or caproic acid and in addition optionally acetic acid is beneficial for therapeutic use in (i) controlling and/or modulating ketosis and/or (ii) controlling and/or modulating ketone body production associated with a ketogenic diet, in a subject adhering to/taking a ketogenic diet, wherein the first composition providing at least one of butyric acid or caproic acid and in addition optionally acetic acid is a composition comprising at least one of butyric acid or caproic acid and in addition optionally acetic acid in non-ketogenic amounts, or a composition comprising a dietary fibre blend providing in non-ketogenic amounts the at least one of butyric acid or caproic acid and in addition optionally acetic acid, wherein the therapeutic use involves a) administering the first composition, c) optionally administering a second composition comprising propionic acid provided that blood ketone levels are above 3 mmol/L. Preferably the therapeutic use involves b) measurement of blood and/or urine ketone levels.

Also, the invention pertains to propionic acid for therapeutic use in i) controlling the effect of a ketogenic diet and/or ii) controlling ketone body production in a subject adhering to a ketogenic diet and using a first composition providing at least one of butyric acid or caproic acid and in addition optionally acetic acid.

Worded differently, the invention pertains to the use of a first composition providing at least one of butyric acid or caproic acid and optionally acetic acid and a second composition comprising propionic acid in the manufacture of a product for (i) controlling and/or modulating ketosis and/or (ii) controlling and/or modulating ketone body production associated with a ketogenic diet, in a subject adhering to/taking a ketogenic diet.

Also, the invention pertains to a (non-therapeutic) method of (i) controlling and/or modulating ketosis and/or (ii) controlling and/or modulating ketone body production associated with a ketogenic diet, in a subject adhering to/taking a ketogenic diet comprising administering to the subject effective amounts of a first composition providing at least one of butyric acid or caproic acid and optionally acetic acid and/or a second composition comprising propionic acid.

The present invention further provides for a kit of parts comprising 1) a ketogenic diet composition having a ketogenic weight ratio between 1:1 and 4:1; 2) a first composition providing at least one of butyric acid or caproic acid and optionally acetic acid; 3) a second composition comprising propionic acid.

There is also provided for a kit of parts for use in (i) controlling and/or modulating ketosis and/or (ii) controlling and/or modulating ketone body production associated with a ketogenic diet in a subject adhering to a ketogenic diet comprising
1) a ketogenic diet composition having a ketogenic weight ratio between 1:1 and 4:1;
2) a first composition providing at least one of butyric acid or caproic acid and optionally acetic acid;
3) a second composition comprising propionic acid.

### LIST OF FIGURES

The present invention will be discussed in more detail below, with reference to the attached figure.

FIGS. 1A-1D: Illustrate the effect of C2 fatty acids on ketone production by liver cells.

FIGS. 2A-2D: Illustrate the effect of C4 fatty acids on ketone production by liver cells.

FIGS. 3A-3D: Illustrate the effect of C6 fatty acids on ketone production by liver cells.

FIGS. 4A-4D: Illustrate the effect of citrate on ketone production by liver cells.

FIGS. 5A-5C: Illustrate the effect of nicotinamide riboside on cellular NAD content.

FIGS. 6A-6C: Illustrate the effect of nicotinamide riboside on ketone production by liver cells.

FIGS. 7A-7D: Illustrate the effect of C3 fatty acids on ketone production by liver cells.

FIGS. 8A and 8B: Illustrate the effect of increased doses of C3 fatty acids on cell viability FIGS. 9A and 9B: Illustrate the effect of a combination of C4 and C6 fatty acids on ketone production by liver cells.

FIG. 10: Illustrates the effect of a combination of C4, C6, citrate and either NR or NAM on ketone production by liver cells.

FIG. 11: Illustrates butyrate to propionate weight ratio of 24-hour fermentation of dietary fibers.

### LIST OF EMBODIMENTS

1. A first composition providing at least one of butyric acid or caproic acid and in addition optionally acetic acid for use in
controlling ketosis associated with a ketogenic diet in a subject adhering to a ketogenic diet,
wherein the at least one of butyric acid or caproic acid and in addition optionally acetic acid is comprised in non-ketogenic amounts in the first composition, or the at least at least one of butyric acid or caproic acid and in addition optionally acetic acid is provided in non-ketogenic amounts by a butyrogenic dietary fiber or a butyrogenic dietary fiber blend comprised in the first composition,
wherein the therapeutic use involves
a) administering the first composition to the subject,
c) optionally administering a second composition comprising propionic acid to reduce ketone production/administering a second composition comprising propionic acid provided that blood ketone levels are above 3 mmol/L.
2. The first composition for use according to embodiment 1 wherein the therapeutic use further involves b) measurement of blood and/or urine ketone levels.
3. The first composition for use according to embodiments 1 and 2 wherein the first and second composition are for sequential use, optionally more than one sequential use.
4. The first composition for use according to any of the preceding embodiments wherein the subject adhering to a ketogenic diet is suffering from a condition selected from the group of conditions consisting of
i) neurological diseases including epilepsy, stroke, traumatic brain injury, migraine;
ii) neurodegenerative diseases including Alzheimer's disease, Parkinson's disease and age-related mild cognitive impairment (MCI);
iii) neuromuscular diseases including amyotrophic lateral sclerosis, ageing-induced muscle inactivation, muscle wasting and sarcopenia, or elderly at risk of developing or suffering from frailty;
iv) psychiatric diseases including attention deficit hyperactivity disorder (ADHD) and autism spectrum disorders;
v) metabolic conditions including, insulin resistance, type-1 and type-2 diabetes mellitus, Glucose transporter type 1 (Glut1) deficiency, Pyruvate dehydrogenase (PDH) deficiency, glycogen storage diseases and mitochondrial diseases;
vi) oncological disorders;
vii) cardiovascular diseases leading to heart failure.
5. First composition for use according to any of the preceding embodiments wherein the first composition further comprises one or more compounds selected from citrate, nicotinamide riboside and nicotinamide.
6. First composition for use according to any the preceding embodiments wherein the first composition comprises
(i) butyric acid
(ii) caproic acid
(iii) citrate, and
(iv) nicotinamide riboside or nicotinamide.
7. First composition for use according to any of the preceding claims wherein the first composition is administered at a dose of
(i) 0.2 mg to 12 mg per kilogram bodyweight per day of butyric acid
(ii) 6 mg to 165 mg per kilogram bodyweight per day of caproic acid
(iii) 0.01 gram to 0.4 gram per kilogram bodyweight per day of citrate, and
(iv) 0.5 mg to 15 mg per kilogram bodyweight per day of nicotinamide riboside.
8. First composition for use according to any of the preceding embodiments wherein the first composition enhances ketosis in a subject adhering to a ketogenic diet.
9. First composition for use according to any of the preceding embodiments wherein the second composition (i) decreases ketone production and/or
(ii) prevents and/or treats ketoacidosis,
wherein (i) decreasing ketone production and/or (ii) preventing and/or treating ketoacidosis includes lowering blood ketone levels below 3 mM.
10. Propionic acid for therapeutic use in
i) controlling the effect of a ketogenic diet and/or
ii) controlling ketone body production
in a subject adhering to a ketogenic diet and using a first composition providing at least one of butyric acid or caproic acid and optionally acetic acid wherein the at least one of butyric acid or caproic acid and in addition optionally acetic acid is comprised in non-ketogenic amounts in the first composition, or the at least at least one of butyric acid or caproic acid and in addition optionally acetic acid is provided in non-ketogenic amounts by a butyrogenic dietary fiber or a butyrogenic dietary fiber blend comprised in the first composition.
11. Propionic acid for therapeutic use according to embodiment 10 wherein i) controlling the effect of a ketogenic diet and/or ii) controlling ketone body production involves lowering blood ketone bodies to a range between 1.0 to 3.0 mmol/L.
12. Propionic acid for therapeutic use according to embodiments 10 and 11 wherein the therapeutic use involves
a) administering the first composition,
b) administering a second composition comprising propionic acid to reduce ketone production/administering a second composition comprising propionic acid.
13. Propionic acid for therapeutic use according to embodiment 12 wherein the therapeutic use further involves c) measurement of blood and/or urine ketone levels.
14. Kit of parts comprising:
1) a ketogenic diet composition having a ketogenic weight ratio between 1:1 and 4:1;
2) a first composition providing at least one of butyric acid or caproic acid and optionally acetic acid wherein the at least one of butyric acid or caproic acid and in addition optionally acetic acid is comprised in non-ketogenic amounts in the first composition, or the at least at least one of butyric acid or caproic acid and in addition optionally acetic acid is provided in non-ketogenic amounts by a butyrogenic dietary fiber or a butyrogenic dietary fiber blend comprised in the first composition;
3) a second composition comprising propionic acid.
15. Kit of parts for use in (i) controlling and/or modulating ketosis and/or (ii) controlling and/or modulating ketone body production associated with a ketogenic diet in a subject adhering to a ketogenic diet comprising:
1) a ketogenic diet composition having a ketogenic weight ratio between 1:1 and 4:1;
2) a first composition providing at least one of butyric acid or caproic acid and optionally acetic acid wherein the at least one of butyric acid or caproic acid and in addition optionally acetic acid is comprised in non-ketogenic amounts in the first composition, or the at least at least one of butyric acid or caproic acid and in addition optionally acetic acid is provided in non-ketogenic amounts by a butyrogenic dietary fiber or a butyrogenic dietary fiber blend comprised in the first composition;
3) a second composition comprising propionic acid.

### DETAILED DESCRIPTION OF THE INVENTION

The inventors have observed that after administration of a first composition providing at least one of butyric acid or caproic acid and optionally acetic acid, the level of ketone bodies produced from a ketogenic diet is enhanced and that after administration of a second composition comprising propionic acid ketone body production is decreased. The inventors have found that the use of the first and the second composition provides a strategy to control and/or modulate blood ketone levels and obtain or maintain a state of ketosis.

It was found by the inventors that the use of butyric acid, caproic acid and optionally acetic acid, when used in amounts that give rise to sub-physiological to physiological plasma levels, increases or enhances the production of ketones of a ketogenic diet while not being a metabolic substrate or precursor for the production of ketone bodies themselves. It was also observed that butyric acid and caproic acid and optionally one or more of acetic acid, citrate and/or nicotinamide riboside and/or nicotinamide have a synergistic enhancing effect on ketosis.

It was found by the inventors that a second composition comprising propionic acid dose-dependently reduces ketone production. It was also found that a second composition comprising propionic acid further provides for lowering of plasma ketone levels below a concentration of 3 mM and treat and/or prevent a blood pH below 7.4. The present inventors have now observed that propionic acid is able to decrease ketone synthesis at low physiological concentrations of 1 μM to 10 μM. Without being bound by theory it is postulated that the use of propionate reduces fatty acid oxidation either by a mechanism comparable to malonyl-CoA (which is an inhibitor of carnitine palmitoyl transferase 1, a protein that mediated the uptake of long chain fatty acids into the mitochondria) or by a conversion through an unknown mechanism of methylmalonyl-CoA, derived from propionate, into malonyl-CoA.

The inventors thus found that a first composition providing at least one of butyric acid or caproic acid and optionally acetic acid is beneficial for therapeutic use in (i) controlling and/or modulating ketosis and/or (ii) controlling and/or modulating ketone body production associated with a ketogenic diet, in a subject adhering to/taking a ketogenic diet, wherein the first composition providing at least one of acetic acid or butyric acid is a composition comprising at least one of butyric acid or caproic acid and optionally acetic acid in non-ketogenic amounts, or a composition comprising a dietary fibre blend providing in non-ketogenic amount the at least one of butyric acid or caproic acid and optionally acetic acid, wherein the therapeutic use involves a) administering the first composition, c) optionally administering a second composition comprising propionic acid provided that blood ketone levels are above 3 mmol/L.

Also, the invention pertains to propionic acid for therapeutic use in i) controlling the effect of a ketogenic diet and/or ii) controlling ketone body production in a subject adhering to a ketogenic diet and using a first composition providing at least one of butyric acid or caproic acid and optionally acetic acid.

Worded differently, the invention pertains to the use of a first composition providing at least one of butyric acid or caproic acid and optionally acetic acid and a second composition comprising propionic acid in the manufacture of a product for (i) controlling and/or modulating ketosis and/or (ii) controlling and/or modulating ketone body production associated with a ketogenic diet, in a subject adhering to/taking a ketogenic diet.

Also, the invention pertains to a method of (i) controlling and/or modulating ketosis and/or (ii) controlling and/or modulating ketone body production associated with a ketogenic diet, in a subject adhering to/taking a ketogenic diet comprising administering to the subject therapeutically effective amounts of a first composition providing at least one of butyric acid or caproic acid and optionally acetic acid and/or a second composition comprising propionic acid.

The present invention further provides for a kit of parts comprising 1) a ketogenic diet composition having a ketogenic weight ratio between 1:1 and 4:1; 2) a first composition providing at least one of butyric acid or caproic acid and optionally acetic acid; 3) a second composition comprising propionic acid.

There is also provided for a kit of parts for use in (i) controlling and/or modulating ketosis and/or (ii) controlling and/or modulating ketone body production associated with a ketogenic diet in a subject adhering to a ketogenic diet comprising
1) a ketogenic diet composition having a ketogenic weight ratio between 1:1 and 4:1;
2) a first composition providing at least one of butyric acid or caproic acid and optionally acetic acid;
3) a second composition comprising propionic acid.

### Definitions

Throughout this application, the following terminology and abbreviations may be used.

A classical ketogenic diet comprises an amount of lipids (by weight), which may be up to about 4-fold the weight of the sum of proteins and digestible carbohydrates. In the context of the invention, the so-called ketogenic (weight) ratio is the weight ratio of the amount of lipid to the combined weight amounts of protein and digestible carbohydrates in the composition. The ketogenic diet referred to in the context of the invention is a dietary intervention that may be any type of ketogenic diet known in the art, preferably characterized by a ketogenic weight ratio between 1:1 and 4:1 which is the ratio of the amount of fat to the combined amounts of protein and digestible carbohydrates. Within the aforementioned (sub)ranges of ketogenic ratios of the invention, the ketogenic diet used within the context of the invention preferably comprises a lipid weight content that is at least twice the carbohydrate weight content. A ketogenic diet composition as used in the context of the invention induces, when adhered thereto, a state of ketosis.

The term “ketosis” as used herein preferably refers to a subject having blood ketone levels above 0.5 mmol/L. Ketone levels sustained above 0.5 mmol/L and ideally in the range of 1 to 3 mmol/L appear to offer therapeutic effects in humans [Anderson J C et al. Obes Sci Pract. 2021; 7(5):646-656)]. Hence, the subject administered with the compositions of the invention preferably exhibits ketone levels in the aforementioned range. Levels of ketones in the blood above 10 mmol/L are associated with signs of ketoacidosis. While ketosis refers to a state of elevated ketones, ketoacidosis is a pathological and potentially life-threatening condition amongst others resulting in a decrease in blood pH below 7.4 and may induce a coma. Means to detect blood and/or urine ketone levels and or blood pH are known in the art and include testing strips, portable blood ketone analysers, enzymatic assays and the like.

In the context of the invention a subject adhering to or taking a ketogenic diet is a mammal, preferably a human.

Fibers are non-digestible carbohydrates. Non-digestible carbohydrates are carbohydrates that are resistant to digestion and absorption in the human stomach and small intestine and enter the colon intact. So, compounds like lactose, maltose, glucose, standard maltodextrin, and standard starch are regarded as digestible. Fibers can be fermentable in the colon, or non-fermentable. The term “fermentable” refers to the capability to undergo (anaerobic) breakdown by micro-organism in the lower part of the gastro-intestinal tract, e.g., colon, to smaller molecules, in particular short chain fatty acids and lactate. The fermentability may be determined by the method described in Am. J. Clin. Nutr. 53, 1418-1424 (1991).

Butyrogenic dietary fibers are fermentable dietary fibers. Butyrogenic dietary fibers or blends produce more hexanoate and butyrate than propionate and pentanoate SCFA. For the sake of this specification, the butyrogenic dietary fibers for use in the present invention are defined as dietary fibers or dietary fiber blends that demonstrate to produce butyrate and propionate in a beneficial ratio. Upon fermentation of butyrogenic dietary fibers the production of pentanoate and hexanoate is negligible,

The term “controlling” and “modulating” as used herein refers to the effect of the first composition and the second composition according to the invention to keep a subject in a therapeutic state of ketosis wherein blood ketone body levels are ideally in the range of 1.0 to 3.0 mmol/L.

The first composition is a composition or combination of compounds which does not increase or enhance the production of ketones by itself (i.e., it is a non-ketogenic composition), but it improves and/or enhances (boosts) the production of the amount of ketones induced by a ketogenic diet. The first composition thus increases (‘boosts’) the ketone amount production of subjects on a ketogenic diet and allows to sustain ketosis and preferably also increases the rate of ketone production without serving as or being a substrate for the production of ketone bodies at the provided amounts. The increase in ketone body production by the first composition in a subject consuming a ketogenic diet or ketogenic substrate occurs with respect to the ketone body production with the same amount of ketogenic diet or ketogenic substrate yet in the absence of the first composition.

The first composition is thus not a metabolic substrate for the production of ketone bodies itself, i.e. the composition is non-ketogenic, but increases or enhances the production of ketone bodies of a ketogenic composition or ketogenic diet. Optionally a compound in the first composition may sometimes give rise to minor amounts of ketone bodies that in itself do not increase plasma ketone concentrations and/or cannot lead to ketosis at the dose provided yet stimulates the formation of ketone bodies of a ketogenic composition in a more than additive manner. The first composition does not have a ketogenic ratio of between 1:1 and 4:1. The first composition may be incorporated within a ketogenic diet or may be a separate composition.

The level of ketone bodies can in turn be balanced and lowered to a blood ketone body level within the desired therapeutic ketosis range by the second composition that dose-dependently allows to reduce ketone production and thereby prevents and/or treats the occurrence of too high levels of ketone bodies and/or ketoacidosis. Together or subsequently the first and second composition allow to control or modulate blood ketone body levels. Where amounts of the first and second composition are provided per kilogram bodyweight this refers to the bodyweight of the subject adhering to/taking a ketogenic diet.

The expression “increasing ketone body production” means that the amount of systemic and/or blood ketone bodies is higher in an individual on i) a ketogenic diet fed (co-administered with) ii) the first composition according to the invention, in comparison with a similar amount and type of ketogenic diet yet without the first composition. In one aspect the first composition decreases the time until therapeutically efficacious ketone concentrations are reached.

The term “decreasing ketone body production” means that the amount of systemic and/or blood ketone bodies is decreased in an individual adhering to a ketogenic diet and fed the second composition according to the invention while consuming a similar amount and type of ketogenic diet. In one aspect the second composition is not a metabolic substrate for the formation of ketone bodies nor a substrate in glucose metabolism.

The expression “non-ketogenic composition” as used herein refers to a composition that upon administration does not lead to the formation of ketone bodies on its own or cannot increase the amount of systemic and/or blood ketone bodies on its own. i.e. it is not a metabolic substrate for the formation of ketone bodies and/or may optionally give rise to the production of minor amounts of ketone bodies that in itself cannot lead to ketosis, yet stimulates the formation of ketone bodies in the presence of a ketogenic diet in a more than additive manner. Non-ketogenic amounts as used herein thus indicate amounts of a compound wherein such a compound in itself does not serve as a substrate for, or lead to the formation of more than just minimal amounts of ketone bodies. Accordingly, the amount of systemic and/or blood ketone bodies do not rise in reaction thereto.

In contrast a ketogenic composition is a composition that may be used to promote the formation of ketone bodies and ketosis in a subject, wherein the composition comprises ketone bodies and/or substrates for the formation of ketone bodies. A ketogenic diet is an exemplary ketogenic composition.

The terms acetic acid or derivative thereof, propionic acid or derivative thereof, butyric acid or derivative thereof and caproic acid or derivative thereof cover respectively any compound that in an animal body is converted into ethanoic acid or ethanoate anion, propionic acid or propanoate anion, butyric acid or butanoate anion and caproic acid or caproate anion respectively. Derivatives include food approved esters, complexes, or salts from the fatty acids. Derivatives of the fatty acids according to the present invention include (partial) glycerol, physiologically acceptable complexes, or salts thereof such as calcium, potassium, sodium, magnesium, ammonium salts and food approved esters.

A glyceride is an ester formed between glycerol and a carboxylic acid, usually in the form of a fatty acid. As an example, a triglyceride (also known as a triacylglycerol) is a triester that is derived from glycerol and three carboxylic acids. Under hydrolysis conditions such as those during digestion, triglycerides may be a source of carboxylic acids or fatty acids. For instance, tributyrin is potentially a source of three moles of butyric acid per mole of tributyrin. Partial glycerides are esters of glycerol with fatty acids, where not all the hydroxyl groups are esterified; mono- and di-butyrin are also sources of butyric acid according to the invention providing for one and two moles of butyric acid per mole respectively.

In the context of the in invention, where reference is made to an amount of ethanoic acid, propionic acid, butyric acid or caproic acid respectively, where different sources or derivatives are used the amounts are calculated in terms of the corresponding (mole) amount.

The term “supplement” or “dietary supplement” refers to a nutritional product that provides nutrients to an individual that may otherwise not conveniently be consumed in sufficient quantities by said individual and may be used to complement the nutrition of an individual. It may be in the form of tablets, capsules, pastilles or a liquid and the like. Supplements typically provide the selected nutrients while not representing a significant portion of the overall nutritional needs of the subject. Typically, they do not represent more than 0.1%, 1%, 5%, 10% of the daily energy need of the subject.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. “Consisting essentially of” means that the first and second composition contains their active ingredients and possibly additional compounds, provided these do not materially affect the essential characteristics of the composition. The indefinite article “a” or “an” thus usually means “at least one”. Where reference is made to % herein it means wt % unless indicated differently.

### Therapeutic Ketosis

An aspect of the present invention is a first composition comprising therapeutically effective amounts of at least one of butyric acid or caproic acid and optionally acetic acid for therapeutic use in (i) controlling and/or modulating ketosis associated with a ketogenic diet and/or (ii) controlling and/or modulating ketone body production associated with a ketogenic diet, in a subject adhering to/taking a ketogenic diet,
wherein the therapeutic use involves,
1) administering said first composition;
2) administering a second composition comprising therapeutically effective amounts of propionic acid provided that blood ketone levels increase above 3 mmol/L,
wherein the first composition and second composition are administered in an amount effective for controlling or modulating ketosis in a subject on a ketogenic diet in the treatment or prevention or improvement of the symptoms of at least a condition selected from the group of conditions consisting of
i) neurological diseases including epilepsy, stroke, traumatic brain injury, migraine;
ii) neurodegenerative diseases including Alzheimer's disease, Parkinson's disease and age-related mild cognitive impairment (MCI);
iii) neuromuscular diseases including amyotrophic lateral sclerosis, ageing-induced muscle inactivation, muscle wasting and sarcopenia, or elderly at risk of developing or suffering from frailty;
iv) psychiatric diseases including attention deficit hyperactivity disorder (ADHD) and autism spectrum disorders;
v) metabolic conditions including overweight, obesity (i.e. a subject having a BMI above 25 or 30 respectively), insulin resistance, type-1 and type-2 diabetes mellitus, Glucose transporter type 1 (Glut1) deficiency, Pyruvate dehydrogenase (PDH) deficiency, glycogen storage diseases and mitochondrial diseases;
vi) oncological disorders;
vii) cardiovascular diseases leading to heart failure.

In a preferred aspect plasma or urine ketone levels are measured using means known in the art such as testing strips and enzymatic methods after 1) administering the first composition and before 2) administering a second composition.

In an aspect of the invention the first composition may also decrease the time until therapeutically efficacious ketone concentrations are reached. A decrease in the time to reach a state of ketosis is of particular use in refractory status epilepticus. In a further aspect of the invention the first composition may also reduce the amount of fat in a ketogenic diet needed to reach and/or maintain a state of ketosis.

In an embodiment the amount of fat in the ketogenic diet may be reduced by about 10% on the basis of a same total amount of calories in the ketogenic diet. By reducing the amount of fat in the ketogenic diet also the ketogenic ratio is reduced by about 10%. It is believed that a reduction in the amount of fats in the ketogenic diet provides for a diet that is easier to comply with and improves patient compliance. The second composition allows to control and/or modulate the blood ketone levels such that blood ketone levels do not become too high, in particular not above 3 mmol/L.

A further aspect of the invention is a second composition comprising therapeutically effective amounts of propionic acid for therapeutic use in (i) controlling and/or modulating ketosis associated with a ketogenic diet and/or (ii) controlling and/or modulating ketone body production associated with a ketogenic diet, in a subject adhering to/taking a ketogenic diet and using a first composition providing at least one of butyric acid or caproic acid and optionally acetic acid wherein the therapeutic use involves lowering and/or controlling blood ketone bodies to a range between 1 to 3 mmol/L and wherein the first composition and second composition are administered in an amount effective for controlling or modulating said blood ketone levels in a subject on a ketogenic diet in the treatment or prevention or improvement of the symptoms of at least a condition selected from the group of conditions consisting of
i) neurological diseases including epilepsy, stroke, traumatic brain injury, migraine;
ii) neurodegenerative diseases including Alzheimer's disease, Parkinson's disease and age-related mild cognitive impairment (MCI);
iii) neuromuscular diseases including amyotrophic lateral sclerosis, ageing-induced muscle inactivation, muscle wasting and sarcopenia, or elderly at risk of developing or suffering from frailty;
iv) psychiatric diseases including attention deficit hyperactivity disorder (ADHD) and autism spectrum disorders;
v) metabolic conditions including overweight, obesity (i.e. a subject having a BMI above 25 or 30 respectively), insulin resistance, type-1 and type-2 diabetes mellitus, Glucose transporter type 1 (Glut1) deficiency, Pyruvate dehydrogenase (PDH) deficiency, glycogen storage diseases and mitochondrial diseases;
vi) oncological disorders;
vii) cardiovascular diseases leading to heart failure.

In one embodiment the second composition comprising propionic acid may also treat and/or prevent the occurrence of ketoacidosis. In one particular embodiment the second composition comprising propionic acid are of particular use in uncontrolled diabetes mellitus and/or in a subject adhering to a ketogenic diet. In one embodiment the subject is starting or building up the use of a ketogenic diet to enter a state of therapeutic ketosis.

The first and second composition according to the invention are not the same compositions, the first composition does not comprise propionic acid and the second composition does not comprise any one of acetic acid, butyric acid, caproic acid, citrate, nicotinamide riboside and nicotinamide.

The subject is preferably a human subject in therapeutic need of ketosis.

### First Composition

### Fatty Acids

Preferably the first composition comprises lipids comprising one or more fatty acids with less than six carbon atoms. Fatty acids which are not attached to other molecules are referred to as free fatty acids (FFA). In a preferred embodiment the fatty acids as used in the present composition are unbranched, more preferably unbranched and even numbered. Most preferably the first composition comprises the fatty acids comprising two (acetic acid or acetate) and/or four (butyric acid or butyrate) and/or six (hexanoic acid and/or hexanoate) carbon atoms. The first composition preferably comprises at least one of butyric acid or caproic acid and optionally acetic acid. Preferably the first composition does not comprise C8 or higher fatty acids.

The fatty acids may be present in the first composition as such in free fatty acid form or as lipids comprising the fatty acids. In the context of the in invention, where reference is made to an amount of fatty acid, where different sources or derivatives are used the amounts are calculated in terms of the corresponding (weight) amount of said short chain fatty acid.

Lipids comprising fatty acids as preferably used in the present invention are preferably selected from the group consisting of triglycerides, diglycerides, monoglycerides, glycolipids, phospholipids and lysophospholipids. In one embodiment the present composition contains triglycerides comprising fatty acyl chains and/or phospholipids comprising fatty acyl chains, more preferably triglycerides comprising fatty acyl chains. The present triglyceride preferably has at least one, more preferably at least two, even more preferably 3 fatty acyl chains.

Lipids comprising fatty acyl chains are degraded by lingual, gastric, duodenal (i.e. pancreatic) and small intestinal lipases. Hence, administration of lipids comprising fatty acyl chains results in the release of fatty acids in the stomach, the duodenum, in the jejunum and ileum.

A preferred source of fatty acyl chains is by chemical synthesis. The source of the lipids may be one or more of animal, plant, fermented, microalgae, GMO, non-GMO or mixtures thereof. An alternative option are lipids obtained from milk from non-human mammals, preferably cow's milk, goat milk, sheep milk, horse milk, buffalo milk, yak milk, reindeer milk, donkey milk and camel milk, particularly cow's milk and/or goat milk. Milk lipid is sometimes also referred to as milk fat or butter fat. In a preferred embodiment, lipids comprising short chain fatty acyl chains are synthesised from glycerol and short chain fatty acids by enzymes such as esterases. Examples are triacetin and tributyrin.

The first composition comprises at least one of butyric acid or caproic acid and optionally acetic acid. In one embodiment when acetic acid is present in the first composition the acetic acid is present in therapeutically effective amount to increase the ketone production of subjects on a ketogenic diet, sustain and/or improve ketosis and/or to prevent or treat tolerance to a ketogenic diet in subjects on a ketogenic diet. In a further embodiment acetic acid are present in therapeutically effective amount for (i) controlling and/or modulating ketosis associated with a ketogenic diet and/or (ii) controlling and/or modulating ketone body production associated with a ketogenic diet.

The ketogenic diet referred to in the context of the invention preferably has a ketogenic weight ratio between 1:1 and 4:1.

Acetic acid is preferably present in the first composition in an amount to provide a daily dosage of acetic acid in the range of 0.5 mg to 30 mg per kilogram bodyweight per day, preferably 1.5 mg to 7.5 mg per kilogram bodyweight per day, more preferably 2.0 mg to 3.0 mg per kilogram bodyweight per day.

In one embodiment acetic acid is present in the first composition as triglyceride form triacetin or glycerol triacetate. Triacetin is, if present, preferably present in the first composition in an amount to provide a daily dosage of triacetin in the range of 0.6 mg to 36 mg per kilogram bodyweight per day, preferably 3 mg to 9 mg per kilogram bodyweight per day, more preferably 2.4 mg to 3.6 mg per kilogram bodyweight per day.

In a particular embodiment the first composition is for use in a subject adhering to a ketogenic diet. In one embodiment when acetic acid is provided in the first composition acetic acid is used and for use together with a ketogenic diet. Acetic acid is preferably provided in a range of 0.005 mg to 0.7 mg acetic acid per kcal of ketogenic diet, more preferably 0.01 mg to 0.4 mg acetic acid per kcal of ketogenic diet, even more preferably 0.02 mg to 0.2 mg acetic acid per kcal of ketogenic diet.

In an embodiment, when butyric acid is present in the first composition, the butyric acid is present in therapeutically effective amount to increases the ketone production of subjects on a ketogenic diet, sustain and/or improve ketosis and/or to prevent or treat tolerance to a ketogenic diet in subjects on a ketogenic diet. In a further preferred embodiment when butyric acid is present in the first composition, the butyric acid is present in therapeutically effective amounts for (i) controlling and/or modulating ketosis associated with a ketogenic diet and/or (ii) controlling and/or modulating ketone body production associated with a ketogenic diet.

Butyric acid is preferably present in the first composition in an amount to provide a daily dosage of butyric acid in the range of 0.2 mg to 12 mg per kilogram bodyweight per day, preferably 1 mg to 6 mg per kilogram bodyweight per day, more preferably 1.5 mg to 3 mg per kilogram bodyweight per day.

In one embodiment butyric acid is present in the first composition as triglyceride form tributyrin or glycerol tributyrate. Tributyrin is, if present, preferably present in the first composition in an amount to provide a daily dosage of tributyrin in the range 0.2 mg to 14 mg per kilogram bodyweight per day, preferably 1 mg to 7 mg per kilogram bodyweight per day, more preferably 2 mg to 5 mg per kilogram bodyweight per day.

In a particular embodiment the first composition is for use in a subject adhering to a ketogenic diet. In one embodiment when butyric acid is provided in the first composition butyric acid is used and for use together with a ketogenic diet. Butyric acid is preferably provided in a range of 0.002 mg to 0.3 mg butyric acid per kcal of ketogenic diet, more preferably 0.015 mg to 0.2 mg butyric acid per kcal of ketogenic diet, even more preferably 0.01 mg to 0.1 mg butyric acid per kcal of ketogenic diet.

### Caproic Acid

In an embodiment the first composition comprises caproic acid.

In an embodiment there is thus provided at least one of caproic acid or butyric acid and in addition optionally acetic acid for therapeutic use in
(i) controlling and/or modulating ketosis associated with a ketogenic diet and/or (ii) controlling and/or modulating ketone body production associated with a ketogenic diet, in a subject adhering to/taking a ketogenic diet, wherein the at least at least one of caproic acid or butyric acid and optionally acetic acid are comprised in non-ketogenic amounts in a first composition, or the at least one of caproic acid or butyric acid and optionally acetic acid are provided in non-ketogenic amounts by a dietary fibre blend comprised in a first composition. Said therapeutic use involves a) administering the first composition, b) optionally administering a second composition comprising propionic acid to reduce ketone production/administering a second composition comprising propionic acid provided that blood ketone levels are above 3 mmol/L.

In an embodiment, when caproic acid is present in the first composition, the caproic acid is present in therapeutically effective amounts to increases the ketone production of subjects on a ketogenic diet, sustain and/or improve ketosis and/or to prevent or treat tolerance to a ketogenic diet in subjects on a ketogenic diet. Caproic acid, when present, allows for
(i) controlling and/or modulating ketosis associated with a ketogenic diet and/or (ii) controlling and/or modulating ketone body production associated with a ketogenic diet, in a subject adhering to/taking a ketogenic diet.

Caproic acid is preferably present in the first composition in an amount to provide a daily dosage of caproic acid in the range of 6 mg to 165 mg per kilogram bodyweight per day, preferably 30 mg to 85 mg per kilogram bodyweight per day, more preferably 50 mg to 70 mg per kilogram bodyweight per day.

In one embodiment caproic acid is present in the first composition as triglyceride form trihexanoin or glycerol trihexanoate. Trihexanoin is, if present, preferably present in the first composition in an amount to provide a daily dosage of trihexanoin in the range of 5 mg to 185 mg per kilogram bodyweight per day, preferably 35 mg to 90 mg per kilogram bodyweight per day, more preferably 50 mg to 75 mg per kilogram bodyweight per day.

In one embodiment when caproic acid is provided in the first composition, caproic acid is used and for use together with a ketogenic diet. Caproic acid is preferably provided in a range of 0.16 mg to 1.6 mg caproic acid per kcal of ketogenic diet, more preferably 0.3 mg to 1.2 mg caproic acid per kcal of ketogenic diet, even more preferably 0.6 mg to 0.8 mg caproic acid per kcal of ketogenic diet.

### Dietary Fibers as Source of Fatty Acids

In an alternative embodiment of the invention the source of fatty acids are dietary fibers. In an embodiment acetic acid and butyric acid may be provided by the microbial fermentation of dietary fibers in the colon. In an embodiment the dietary fibers are provided in the first composition in an amount sufficient to provide acetic acid and butyric acid in a therapeutic effective range. In an embodiment there is provided between 1 g and 2 g of dietary fibers per 100 kcal of the total diet.

In a preferred embodiment, dietary fibers in the non-ketogenic ketogenic diet-booster composition serve as a substrate for fatty acid production. The dietary fibers that serve as a substrate for fatty acids in the context of the invention are butyrogenic dietary fibers or butyrogenic dietary fiber blends. Butyrogenic dietary fibers or blends when fermented produce, more hexanoate and butyrate than propionate and pentanoate FA. For the sake of this specification, as the production of pentanoate and hexanoate are negligible anyway, the butyrogenic dietary fibers for use in the present invention are defined as dietary fibers or dietary fiber blends that produce butyrate and propionate in a beneficial ratio A beneficial weight ratio of butyrate to propionate as used herein is defined as more butyrate than propionate, i.e. a butyrate to propionate weight ratio of at least 1, preferably at least 1.1.

The butyrogenic dietary fibers that may serve as a substrate for generating fatty acids, preferably produce acetate, butyrate, propionate, and hexanoate, more preferably propionate and butyrate in a beneficial ratio, allowing to increase the ketone production of subjects on a ketogenic diet, sustain and/or improve ketosis and/or to prevent or treat tolerance to a ketogenic diet in subjects on a ketogenic diet.

The expression “a beneficial butyrate to propionate ratio” as used herein means that the weight of butyrate produced by fermentation of a fiber or mixture of fibers is at least equal or higher that the amount of propionate produced by the fermentation. For example, upon fermentation of locust bean gum or yeast beta-glucan more propionate than butyrate is produced, and these fibers thus have a butyrate to propionate ratio below 1 and cannot boost or increase ketone production. In contrast, soy fibers and inulin are exemplary fibers that upon fermentation have a beneficial butyrate to propionate ratio.

In a preferred embodiment the fermentation of butyrogenic dietary fibers beneficially results in a fatty acid production wherein butyrate and propionate are produced in a weight ratio of butyrate:propionate at least 1, preferably at least 1.1. Fermentation profiles of dietary fibers and concomitant butyrate to propionate ratio's from either a single source of fibers or blends of fibers, can be assessed using fermentation assays known in the art. Exemplary ways to assess the fermentation of fibers include using in vitro fermentation models with faecal sample pools of healthy adults with exposure to approximately 10 mg/ml fiber for 24 hours at 37° C. under anaerobic conditions.

In an embodiment there is provided between 1 g and 2 g of dietary fibers per 100 kcal of the total diet. In an embodiment in a diet of about 2500 kcal per day there is provided between 25 and 50 g of dietary fibers that may provide for about 10 μM to about 250 μM of fatty acids including C2, C4 and C6 fatty acids in the blood.

The dietary fibers that provide acetic acid, butyric acid and/or caproic acid are fermentable dietary fibers, such as for example fermentable oligo- and polysaccharides. Preferably said dietary fibers are butyrogenic dietary fibers having a beneficial fermentation profile wherein butyrate and propionate are produced in a weight ratio of C4:C3 of at least 1, preferably at least 1.1. The fermentable fibers can include but are not restricted to pectins, mucilages, gums, galacto-oligosaccharides, oligofructan, inulin, polyfructoses, fructooligosaccharides, arabinogalactans, hemicelullose, oligosaccharides, resistant starch, soy fiber (soluble soy polysaccharides) or mixtures of thereof.

In one embodiment, the butyrogenic dietary fiber serving as a substrate for fatty acid production is a fermentable fiber which is selected in the group consisting of: pectins, mucilages, gums, galacto-oligosaccharides, oligofructan, inulin, polyfructoses, fructo-oligosaccharides, arabinogalactans, (hemi)celullose, resistant starch, soy fiber, oligosaccharides, and mixtures of thereof.

In a particular embodiment the use of fiber blends is preferred. Exemplary fiber blends for use in the context of the present invention are blends that are fermentable and provide a beneficial C4:C3 weight ratio of at least 1, preferably at least 1.1, even more preferably at least 1.5. An exemplary fiber blends is a fibre mixture that comprises a) beta-galactooligosaccharides, b) inulin, c) resistant starch and/or d) soluble soy polysaccharides provide fatty acids, in particular at a beneficial C4:C3 weight ratio.

A further exemplary fiber blend comprises a) FOS, b) inulin, c) soy fibre, d) resistant starch, e) acacia gum and/or f cellulose. The blend is fermentable and provides fatty acids, in particular at a beneficial C4:C3 weight ratio of at least 1, preferably at least 1.1, even more preferably at least 1.5.

A further exemplary fiber blend comprises a) arabinoxylan, b) oat beta-glucan, c) pectin, and/or d) resistant starch. The fiber mixture is fermentable and produces butyrate and propionate in a weight ratio of C4:C3 of at least 1, preferably at least 1.1, even more preferably at least 1.5.

In an embodiment there is thus provided at least one of caproic acid or butyric acid and in addition optionally acetic acid for therapeutic use in
(i) controlling and/or modulating ketosis associated with a ketogenic diet and/or (ii) controlling and/or modulating ketone body production associated with a ketogenic diet, wherein the at least one of caproic acid or butyric acid and in addition optionally acetic acid is comprised in non-ketogenic amount in a first composition, or the at least one of caproic acid or butyric acid and in addition optionally acetic acid is provided in non-ketogenic amounts by a dietary fibre blend comprised in a first composition.

The therapeutic use involves a) administering the first composition, b) optionally administering a second composition comprising propionic acid to reduce ketone production/administering a second composition comprising propionic acid provided that blood ketone levels are above 3 mmol/L.

In a further embodiment there is thus provided at least one of caproic acid or butyric acid and in addition optionally acetic acid for therapeutic use in (i) enhancing ketosis and/or (ii) preventing and/or treating resistance to a ketogenic diet, in a subject adhering to/taking a ketogenic diet, wherein the at least one of caproic acid or butyric acid and in addition optionally acetic acid is comprised in non-ketogenic amount in a first composition, or the at least one of caproic acid or butyric acid and in addition optionally acetic acid is provided in non-ketogenic amounts by a dietary fibre blend comprised in a first composition.

### Citrate

In a further embodiment the first composition may further comprise citric acid or citrate. Citrate is converted to isocitrate in the first step in the Krebs cycle (also known as the citric acid or TCA cycle). Citrate as used in the first composition is preferably in the form of a salt complexed to potassium (K′), sodium (Na⁺), Magnesium (Mg²⁺) or calcium (Ca²⁺). In the context of the invention the amounts are calculated in terms of the corresponding (weight) amount of citrate.

In an embodiment there is thus provided at least one of caproic acid or butyric acid and optionally acetic acid in therapeutically effective amounts of for use in
(i) controlling and/or modulating ketosis associated with a ketogenic diet and/or
(ii) controlling and/or modulating ketone body production associated with a ketogenic diet, in a subject adhering to/taking a ketogenic diet,
wherein the at least one of caproic acid or butyric acid and optionally acetic acid is comprised in non-ketogenic amounts in a first composition, or the at least one of caproic acid or butyric acid and optionally acetic acid is provided in non-ketogenic amounts by a dietary fibre blend comprised in a first composition, wherein the first composition optionally further comprises therapeutically effective amounts of citrate. Said therapeutic use involves a) administering the first composition, b) optionally administering a second composition comprising propionic acid to reduce ketone production/administering a second composition comprising propionic acid provided that blood ketone levels are above 3 mmol/L.

In an embodiment, when citrate is present in the first composition, the citrate is present in a therapeutically effective amounts to increases the ketone production of subjects on a ketogenic diet, sustain and/or improve ketosis and/or to prevent or treat tolerance to a ketogenic diet in subjects on a ketogenic diet. In a further embodiment when citrate is present in the first composition, citrate is present in a therapeutically effective amounts for (i) controlling and/or modulating ketosis associated with a ketogenic diet and/or (ii) controlling and/or modulating ketone body production associated with a ketogenic diet, in a subject adhering to/taking a ketogenic diet.

Citrate is, if present, preferably present in the first composition in an amount to provide a daily dosage of citrate in the range of 0.01 gram to 0.4 gram per kilogram bodyweight per day, preferably 0.04 gram to 0.15 gram per kilogram bodyweight per day, more preferably 0.08 gram to 0.1 gram per kilogram bodyweight per day.

In one embodiment when citrate is provided in the first composition, citrate is used and for use together with a ketogenic diet. Citrate is preferably provided in a range of 0.1 mg to 10 mg citrate per kcal of ketogenic diet, more preferably 0.25 mg to 7 mg citrate per kcal of ketogenic diet, even more preferably 0.4 mg to 3.5 mg citrate per kcal of ketogenic diet.

In an embodiment the first composition comprises butyric acid, caproic acid and citrate.

Daily intake of citrate of less than 15 g/day for K⁺/Na⁺/Mg⁺-citrate and possibly less than 20 g/day for Ca²⁺-citrate is considered safe and does not give rise to a risk of electrolyte imbalances.

### Nicotinamide Riboside

The present disclosure provides for a first composition further comprising a NAD+ precursor selected from the group consisting of tryptophan, nicotinic acid (niacin), nicotinamide (niacinamide), nicotinic acid riboside (NaR), nicotinamide riboside (NR), and mixtures thereof. In a preferred aspect the first composition may comprise nicotinamide riboside (NR and/or nicotinamide).). In the context of the invention the amounts are calculated in terms of the corresponding (weight) amount of nicotinamide riboside.

As used herein, “nicotinamide riboside” includes derivatives thereof such as L-valine and L-phenylalanine esters of nicotinamide riboside.

In an embodiment there is thus provided at least one of caproic acid or butyric acid and optionally acetic acid in therapeutically effective amounts for use in
(i) controlling and/or modulating ketosis associated with a ketogenic diet and/or
(ii) controlling and/or modulating ketone body production associated with a ketogenic diet, in a subject adhering to/taking a ketogenic diet,
wherein the at least one of caproic acid or butyric acid and optionally acetic acid is comprised in non-ketogenic amounts in a first composition, or the at least one of caproic acid or butyric acid and optionally acetic acid is provided in non-ketogenic amounts by a dietary fibre blend comprised in a first composition, wherein the first composition optionally further comprises therapeutically effective amounts of a NAD+ precursor selected from nicotinic acid, nicotinamide riboside and/or nicotinamide, preferably nicotinamide riboside and/or nicotinamide. Said therapeutic use involves a) administering the first composition, b) optionally administering a second composition comprising propionic acid to reduce ketone production/administering a second composition comprising propionic acid provided that blood ketone levels are above 3 mmol/L.

In an embodiment, when nicotinamide riboside or nicotinamide is present in the first composition, the nicotinamide riboside is present in therapeutically effective amounts to increase the ketone production of subjects on a ketogenic diet, sustain and/or improve ketosis and/or to prevent or treat tolerance to a ketogenic diet in subjects on a ketogenic diet. In a further aspect, when nicotinamide riboside or nicotinamide is present in the first composition, the nicotinamide riboside or nicotinamide is present in therapeutically effective amounts for (i) controlling and/or modulating ketosis associated with a ketogenic diet and/or (ii) controlling and/or modulating ketone body production associated with a ketogenic diet, in a subject adhering to/taking a ketogenic diet.

Nicotinamide riboside is, if present, preferably present in the first composition in an amount to provide a daily dosage of nicotinamide riboside in the range of 0.5 mg to 15 mg per kilogram bodyweight per day, preferably 1 mg to 5 mg per kilogram bodyweight per day, more preferably 2.5 mg to 3.5 mg per kilogram bodyweight per day.

In one embodiment when nicotinamide riboside is provided in the first composition, nicotinamide riboside is used and for use together with a ketogenic diet. Nicotinamide riboside is preferably provided in a range of 0.01 mg to 0.4 mg nicotinamide riboside per kcal of ketogenic diet, more preferably 0.015 mg to 0.25 mg nicotinamide riboside per kcal of ketogenic diet, even more preferably 0.02 mg to 0.1 mg nicotinamide riboside per kcal of ketogenic diet.

In an embodiment the first composition comprises butyric acid, caproic acid, citrate and a NAD+ precursor.

### Second Composition Comprising Propionate

Propionic acid or propionate according to the invention may be present as such in free fatty acid form, as lipids comprising the fatty acids or as physiologically acceptable derivatives thereof. Propionic acid is preferably present in a second composition wherein propionic acid is present as active ingredient. In an aspect of the invention such a second composition is consisting essentially of propionic acid.

In an embodiment the second composition comprises therapeutically effective amounts of propionic acid or derivative thereof in the form of lipids comprising propionic acid. Lipids comprising propionic acid are preferably selected from the group consisting of triglycerides, diglycerides, monoglycerides, glycolipids, phospholipids and lysophospholipids. In one embodiment the present composition contains triglycerides comprising fatty acyl chains and/or phospholipids comprising fatty acyl chains, more preferably triglycerides comprising fatty acyl chains. The present triglyceride preferably has at least one, more preferably at least two fatty acyl chains, which is preferably at the sn-3 position. In an embodiment propionate may be present in the form of glycerol derivatives monopropionin, dipropionin or tripropionin, preferably as tripropionin.

Lipids comprising fatty acyl chains are degraded by lingual, gastric, duodenal (i.e. pancreatic) and small intestinal lipases. Hence, administration of lipids comprising fatty acyl chains results in the release of fatty acids in the stomach, the duodenum, in the jejunum and ileum.

In an embodiment the second composition comprises propionic acid in the form of physiologically acceptable salts thereof such as calcium, potassium, sodium, magnesium, ammonium salts of propionate.

In a further embodiment the second composition comprises propionic acid in the form of organic esters of propionic acid, such as for example methyl, ethyl, propyl or iso-propyl esters such as ethyl propionate.

Such esters may be naturally occurring or may be obtained via chemical reactions for example by condensation of a SCFA and an appropriate alcohol (for example ethylic alcohol).

Propionic acids are provided in an amount to provide a daily dosage in the range of 1 mg to 1200 mg per day, preferably 5 mg to 600 mg per day, more preferably 10 mg to 400 mg per day of propionic acid.

In an embodiment propionic acid is provided in triglyceride form as tripropionin or glycerol tripropanoate in an amount to provide a daily dosage in the range of 1 mg to 1400 mg per day, preferably 6 to 700 mg per day, more preferably 12 to 500 mg per day.

Alternatively, the propionic acid is provided in diglyceride form as dipropionin or glycerol dipropanoate in an amount to provide a daily dosage in the range of 1 mg to 1700 mg per day, preferably 5 to 1000 mg per day, more preferably 15 to 600 mg per day.

In a further alternative embodiment, the propionic acid is provided in monoglyceride form as monopropionin or glycerol monopropanoate in an amount to provide a daily dosage in the range of 2 mg to 2400 mg per day, preferably 10 to 1200 mg per day, more preferably 20 to 800 mg per day.

In an embodiment mixtures of mono-, di- and/or tripropionin provide the propionic acid according to the invention.

In a particular embodiment a second composition comprising propionic acid is for use in a subject adhering to a ketogenic diet. Propionic acid is used and for use together with a ketogenic diet. Propionic acid is preferably provided in a range of 0.002 mg to 0.3 mg propionic acid per kcal of ketogenic diet, more preferably 0.01 mg to 0.15 mg propionic acid per kcal of ketogenic diet, even more preferably 0.02 mg to 0.1 mg propionic acid per kcal of ketogenic diet.

Alternatively, propionic acid is provided in triglyceride form as tripropionin or glycerol tripropanoate in a range of 0.002 mg to 0.4 mg tripropionin per kcal of ketogenic diet, more preferably 0.01 mg to 0.18 mg tripropionin per kcal of ketogenic diet, even more preferably 0.02 mg to 0.1 mg tripropionin per kcal of ketogenic diet.

In a further alternative embodiment propionic acid is provided in diglyceride form as dipropionin or glycerol dipropanoate in a range of 0.003 mg to 0.4 mg dipropionin per kcal of ketogenic diet, more preferably 0.015 mg to 0.2 mg dipropionin per kcal of ketogenic diet, even more preferably 0.02 mg to 0.1 mg dipropionin per kcal of ketogenic diet.

In a further embodiment propionic acid is provided in monoglyceride form as monopropionin or glycerol monopropanoate in a range of 0.004 mg to 0.6 mg monopropionin per kcal of ketogenic diet, more preferably 0.02 mg to 0.3 mg monopropionin per kcal of ketogenic diet, even more preferably 0.06 mg to 0.1 mg monopropionin per kcal of ketogenic diet.

### Administration Forms of the First and Second Composition

The first and second composition of the invention may be in any form suitable for enteral, oral or parenteral administration. Non-limiting examples of parenteral administration include intravenously, intramuscularly, intraperitoneally, subcutaneously, intraarticularly, intrasynovially, intraocularly, intrathecally, topically, and inhalation]

In one aspect of the present invention, the first and second composition according to the invention may be used as a pharmaceutical product comprising one or more pharmaceutically acceptable carrier materials.

The first and second composition according to the invention may in a preferred aspect be used as a nutritional product, for example as a nutritional supplement, e.g., as an additive to a ketogenic diet, as a fortifier, to add to a ketogenic diet.

A supplement, preferably for enteral application, may be a solid or liquid galenical formulation. Examples of solid galenical formulations are tablets, capsules (e.g., hard- or soft-shell gelatine capsules), pills, sachets, powders, granules and the like which contain the active ingredient together with conventional galenical carriers. Any conventional carrier material can be utilized. The carrier material can be organic or inorganic inert carrier material suitable for oral administration. Suitable carriers include water, gelatine, gum Arabic, lactose, starch, magnesium stearate, talc, vegetable oils, and the like. Additionally, additives such as flavouring agents, preservatives, stabilizers, emulsifying agents, buffers, and the like may be added in accordance with accepted practices of nutritional and pharmaceutical compounding.

The first and second composition according to the invention may also be used as a nutritional product for use together with tube feeding, e.g., as an additive to a tube feed for intermittent or optionally continuous administration.

The first and second composition may contain the daily dosage in one or more dosage units. The dosage unit may be in a liquid form or in a solid form, and in both cases the daily dosage may be provided by one or more dosage units, e.g., in one or more drinks, capsules or tablets. The ketogenic booster composition can be administered at the same time as the ketogenic diet or separated by a time interval or continuous.

A subject may receive one or more doses of the first composition daily, preferably 1 to 6 times per day, more preferably 3 to 4 times per day. In some embodiments, the administration continues for the remaining life of the individual. The subject using the first composition may receive one or more doses or servings of the first composition daily, preferably 1 to 6 times per day, more preferably 3 to 4 times per day.

The ideal duration and order of the administration of the first and second composition can be determined by those of skill in the art.

In a particular non-limiting example, the daily doses of a first composition for a 70 kg subject consuming 3000 kcal per day of a ketogenic diet having a ketogenic ratio of 1:1 to 4:1 can be as follows:
Acetic acid or derivative thereof: between 35 mg/day to 2520 mg/day;
Butyric acid or derivative thereof: between 7 mg/day to 980 mg/day;
Caproic acid or derivative thereof: between 455 mg/day to 12.95 g/day;
Citrate: between 0.7 g/day to 20 g/day;
NR: between 42 mg/day to 1015 mg/day.

In a further non-limiting example, the daily dose of a second composition for a 70 kg subject consuming 3000 kcal per day of a ketogenic diet having a ketogenic ratio of 1:1 to 4:1 and using the first composition according to the invention can be as follows:
Propionic acid: between 3000 kcal/day*(0.002 mg to 0.3 mg propionic acid per kcal)=6 mg to 900 mg C3/day.

In a further embodiment there is provided the first composition providing essentially
(i) 1 mg to 1.2 g butyric acid/day; and optionally one or more of
(ii) 32.5 mg to 16.3 g caproic acid/day;
(iii) 0.05 g to 20 g citrate/day;
(iv) 3 mg to 1.45 g nicotinamide riboside/day; and
(v) 2.5 mg to 3 g acetic acid/day.

The ranges refer to the total amounts per 24 hours.

In a further embodiment there is also provided the second composition providing essentially
(i) 1 mg to 1200 mg propionic acid per day. The range refers to the total amounts per 24 hours.

In an embodiment there is provided the first composition according to the invention consisting essentially of
(i) 0.2 mg to 1.2 g butyric acid;
(ii) 5 mg to 17 g caproic acid;
(iii) 10 mg to 20 g citrate; and
(iv) 0.5 mg to 1.5 g nicotinamide riboside per serving based on dry weight of the composition.

In a further embodiment there is provided the first composition consisting essentially of
(i) 0.2 mg to 1.2 g butyric acid;
(ii) 5 mg to 17 g caproic acid;
(iii) 10 mg to 20 g citrate;
(iv) 0.5 mg to 1.5 g nicotinamide riboside; and
(v) 0.5 mg to 3 g acetic acid per serving based on dry weight of the composition.

In an embodiment there is provided the first composition, intended for administration 4 to 6 times per day, consisting essentially of
(i) 0.1-0.3 mg butyric acid;
(ii) 4.6-5.4 mg caproic acid;
(iii) 9-11 mg citrate; and
(iv) 0.4-06 mg nicotinamide riboside per serving. Optionally the first composition intended for administration 4 to 6 times per day additionally consists essentially of (v) 0.4-0.6 mg acetic acid.

In a further embodiment there is provided the first composition, intended for administration once daily, consisting essentially of
(i) about 1.2 g butyric acid;
(ii) about 17 g caproic acid;
(iii) about 20 g citrate; and
(iv) about 1.5 g nicotinamide riboside per serving. Optionally the first composition intended for administration once daily additionally consists essentially of (v) about 3 g acetic acid.

Typically, a serving of the first composition is between about 10 g and 25 g.

In a further embodiment there is provided the first composition according to the invention consisting essentially of
(i) 20 mg to 1.2 g acetic acid;
(ii) 4 mg to 480 mg butyric acid;
(iii) 260 mg to 3.3 g caproic acid;
(iv) 0.2 mg to 4.4 g citrate; and
(v) 12 mg to 286 mg nicotinamide riboside,
wherein the weight ratio of acetic acid and butyric acid:caproic acid is between 1:1.8 and 1:2.1, preferably between 1:1.9 and 1:2.0;
the weight ratio of acetic acid and butyric acid:citrate is between 1:2.4 and 1:2.7, preferably between 1:2.5 and 1:2.6 and
the weight ratio of acetic acid and butyric acid:nicotinamide riboside is between 1:0.15 and 1:0.19, preferably between 1:0.16 and 1:0.18 based on dry weight and wherein the total weight of the composition is between about 10-20 grams.

Said first and second compositions are formulated in the form of a dietary supplement to a ketogenic diet.

Further provided herein are kits of parts.

Provided is a kit of parts comprising:
1) a ketogenic diet composition having a ketogenic weight ratio between 1:1 and 4:1;
2) a first composition providing at least one of caproic acid or butyric acid and optionally acetic acid;
3) a second composition comprising propionic acid.

The kit of part optionally further comprises 4) means for measuring ketone level in a body fluid.

Also provided herein is a kit of parts for use in (i) controlling and/or modulating ketosis and/or (ii) controlling and/or modulating ketone body production associated with a ketogenic diet in a subject adhering to a ketogenic diet comprising:
1) a ketogenic diet composition having a ketogenic weight ratio between 1:1 and 4:1;
2) a first composition providing at least one of caproic acid or butyric acid and optionally acetic acid;
3) a second composition comprising propionic acid.

The kit of part for use in (i) controlling and/or modulating ketosis and/or (ii) controlling and/or modulating ketone body production associated with a ketogenic diet in a subject adhering to a ketogenic diet comprising optionally further comprises 4) means for measuring ketone level in a body fluid.

It is understood that a kit of parts is a range of compositions according to the invention conceptually belonging together. The individual components of the kit do not have to be packaged together.

### EXAMPLES

For a more complete understanding of the present disclosure, reference is now made to the following examples taken in conjunction with the accompanying drawings.

### Example 1

An assay to measure ketone production in mouse liver cells (Hepa1-6 cell line) was used wherein cells were first grown to a monolayer (growth phase), then exposed to starvation to deplete cellular lipid stores and subsequently brought into a ketogenic phase wherein cells were cultured in presence of oleic acid or a ketogenic diet fat blend with or without the presence of one of C2, C4, C6 fatty acids, citrate and nicotinamide riboside (herein also referred to as C2, C4, C6, citrate and NR). The ketone body production was subsequently determined by measurement of beta-hydroxybutyrate in the cell culture medium according to the details set out below:

Growth phase: Cells were seeded in 6-well plates at a concentration of 90700 cells/cm² and allowed to grow for 24 hours at 37° C. in Dulbecco's Modified Eagle Medium (DMEM) supplemented with 5 mM glucose, 10% Fetal Bovine serum (FBS), 4 mM L-glutamine, 1 mM pyruvate, and antibiotics (1% penicillin & streptomycin).

Starvation phase: At the end of the growth phase the cells formed a monolayer. The medium was exchanged for DMEM containing 1 mM glucose and antibiotics (1% penicillin & streptomycin) but no FBS. The medium was further supplemented with L-carnitine (a required co-factor for the uptake of fatty acids into the mitochondria, where fatty acids are turned into ketones). The cells were incubated in this medium for 24 hours. During this time, the cells were vastly deprived of glucose, lipids and hormones contained in FBS, forcing them to consume and empty their lipid stores. Thereby, any interference with the nutrient testing was avoided. Moreover, the starvation phase is thought to induce specific metabolic changes that maximize the production of ketones.

Ketogenic phase: At the end of the starvation phase, the medium was exchanged for Krebs-Henseleit buffer (KHB) supplemented with the test nutrients (single fatty acids and fatty acid blend shown in table 1) and L-carnitine.

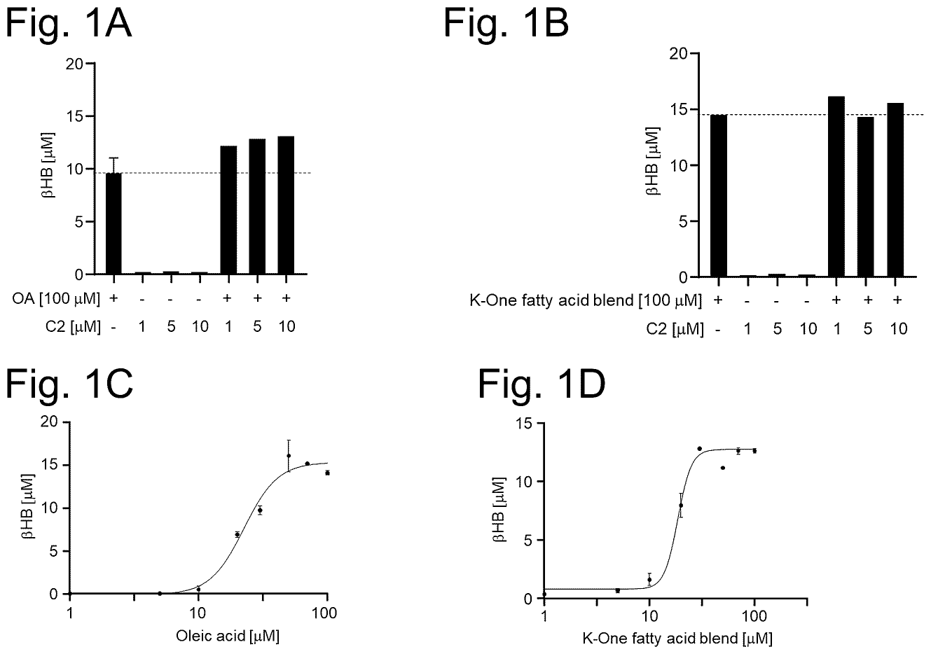

The fatty acid blend is contained in a similar fatty acid composition to the one used in a ketogenic diet (K-one). The content of every fatty acid in the blend is given as the molar ratio to Oleic acid (OA), whose molar content is set to 1. Concentrations of individual fatty acids in 100 μM of the blend are shown in Table 1. Dilutions of this blend were used to determine the dose response curve in FIGS. 1B and 1D. C2 and C4 are not included in the blend.

TABLE 1
Composition of fatty acid blend.

| | | | Concentration (μM) |
|---|---|---|---|
| | Lipid | molar | in 100 μM total |
| Ingredient | numbers | ratio | fatty acid blend. |
| caproic acid | C-6:0 | 0.0015 | 0.05 |
| caprylic acid | C-8:0 | 0.0463 | 1.72 |
| capric acid | C-10:0 | 0.0363 | 1.35 |
| lauric acid | C-12:0 | 0.1071 | 3.98 |
| myristic acid | C-14:0 | 0.0682 | 2.53 |
| palmitic acid | C-16:0 | 0.9354 | 34.73 |
| stearic acid | C18:0 | 0.1184 | 4.39 |
| oleic acid | C18:1ω9 | 1.0000 | 37.13 |
| linoleic acid | C18:2ω6 | 0.2618 | 9.72 |
| alpha-linolenic acid | C18:3ω3 | 0.0274 | 1.02 |
| eicosapentaenoic acid | C20:5ω3 | 0.0168 | 0.62 |
| docosahexaenoic acid | C22:6ω3 | 0.0741 | 2.75 |

KHB is composed of salts and bicarbonate as a chemical buffer to maintain the osmotic pressure and acidity (pH) at physiological levels. Medium and long chain fatty acids are coupled to bovine serum albumin prior to the assay to make them soluble in KHB and allow their efficient uptake by the cells. The coupling is physiological since in humans, medium and long chain fatty acids are also normally coupled to albumin for transport in the blood circulation. Short chain fatty acids (C2, C4) were not coupled to albumin in the assay since they are water-soluble and circulate freely in humans. The cells were incubated for 6 hours at 37° C. During this time, they produce ketones from the test nutrients with little to no interference from other nutrients and secrete β-hydroxybutyrate (β-Hb) into the medium.

β-Hb measurements: At the end of the ketogenic phase, the whole medium was collected and dried by vacuum centrifugation for 20 hours at 21° C. The dry pellet was resuspended in a small volume of assay buffer to yield a 10-20-fold higher β-Hb concentration than in the medium. The samples were deproteinized using spin columns to measure the free unbound fraction of R-Hb. The β-Hb concentration was then measured by an enzymatic reaction that generates a fluorescent product (Cayman Chemicals 700740). The Fluorescence was measured on a fluorescence microplate reader and is directly proportional to the β-Hb concentration in the sample within a range of 0.1 μM to 50 μM β-Hb.

### 1.1 Effect of C2 on Ketone Production

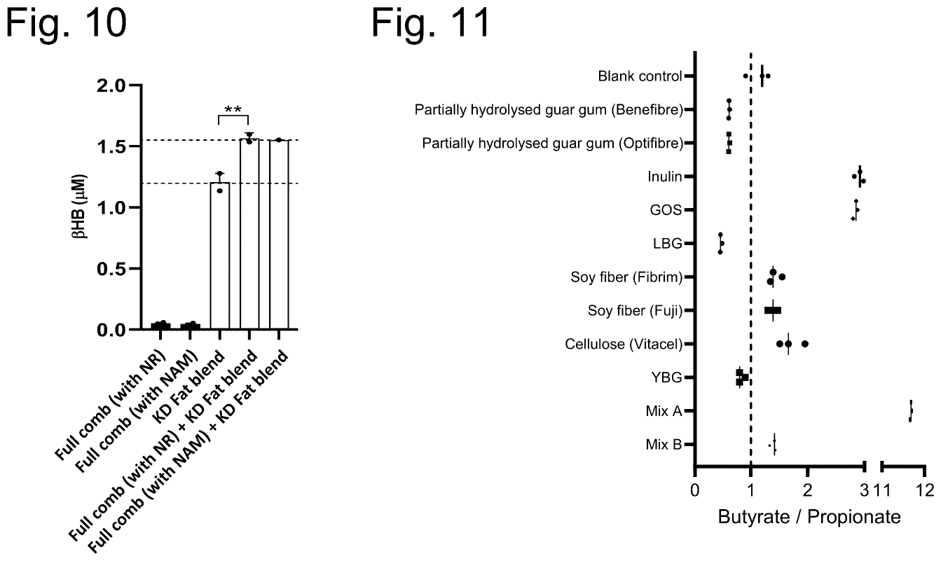

In the above ketone production assay, 02 provided alone to liver cells at a concentration of 1 μM, 5 μM and 10 μM yielded virtually no bHB (FIGS. 1A and 1B). When liver cells were incubated with a concentration range of oleic acid (OA), a long chain fatty acid, the dose-response curve showed that the top-level production of ketones from oleic acid (OA) alone was achieved at a concentration of 100 μM OA (FIG. 10C). The fatty acid plasma concentrations during a ketogenic diet treatment are usually above 100 μM in humans. Therefore, to assess the ketogenic enhancement by C2, OA was assayed at 100 μM. Combining 1 μM, 5 μM, or 10 μM C2 with 100 μM OA yielded more PHb than 100 μM OA alone. The effect of combining C2 and OA on PHb production was more than additive, it cannot be explained by the sum of the individual effects of C2 and OA on PHb production. Therefore, C2 at a physiological concentration of 1 μM to 10 μM potentiates the top-level production of ketones from a single fatty acid. The same experiment was performed with a fatty acid blend composed of medium, long and very long chain fatty acids and contained in a ketogenic diet (denoted as K-one). The exact composition of the blend is shown in table 1 and does not contain C2. When liver cells were incubated with a concentration range of this blend, the dose-response curve showed that production of PHb from this blend was maximized at a total fatty acid concentration of 100 μM (FIG. 1D). The fatty acid concentrations of humans on a ketogenic diet in plasma exceeds 100 μM, meaning that the top-level production of ketones is then achieved. Therefore, to assess the ketogenic enhancement by C2, the fatty acid blend was tested at a concentration of 100 μM. Combining 1 μM or 10 μM C2 with 100 μM fatty acid blend led to slightly greater production of PHb than an equimolar concentration of the fatty acid blend alone (FIG. 1B). The effect of combining C2 and the fatty acid blend on PHb production was more than additive since C2 alone yielded no PHb (FIG. 1B). Therefore, in a physiological concentration range of 1 μM to 10 μM, C2 potentiates the top-level production of PHb from a fatty acid blend contained in a ketogenic diet.

To summarize, C2 within a low physiological concentration of 1 μM to 10 μM enhances the production of ketones (PHb) from a single fatty acid (oleic acid, OA) (FIG. 1A) and from a blend of fatty acids contained in a ketogenic diet (K-one) (FIG. 1B). The effect of C2 is not explained by it being used as a substrate since it does not yield any ketones when provided alone to liver cells. Furthermore, the single fatty acid (oleic acid) and the fat blend (K-one) were tested with C2 at a concentration of 100 μM, when they both maximize ketone production on their own (FIGS. 1C, D). Therefore, C2 potentiates the top level production of ketones from a single fatty acid or a blend of fatty acids.

### 1.2 Effect of C4 on Ketone Production

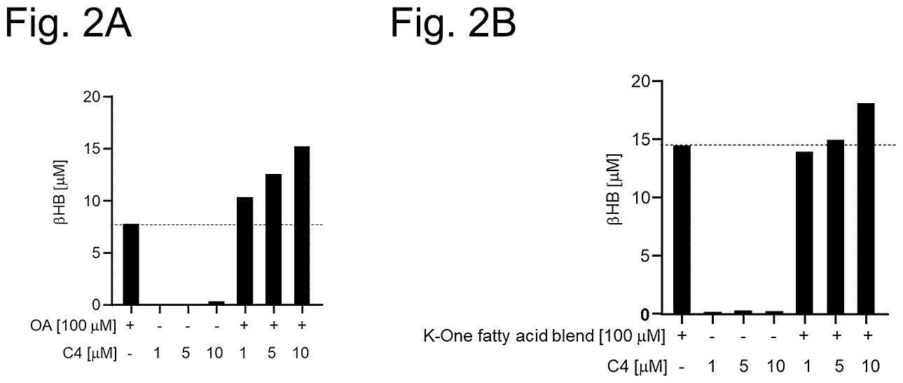

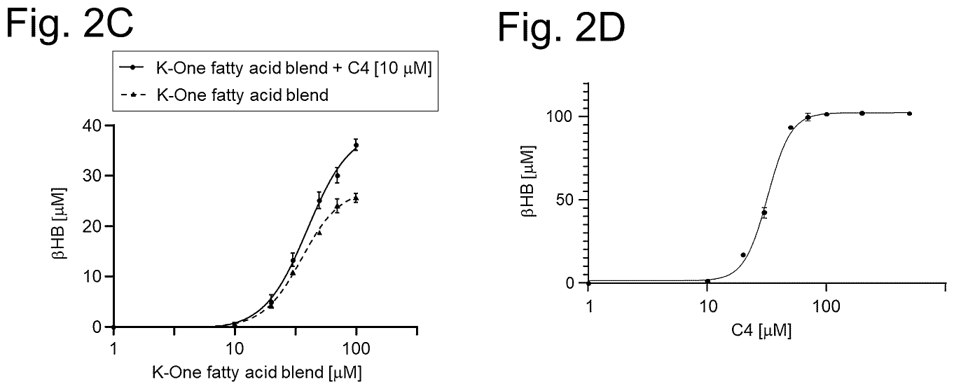

In the above ketone production assay, exposing liver cells to C4 alone at a concentration of 1 μM, 5 μM and 10 μM hardly yielded any PHb (FIGS. 2A and 2B). The top level production of ketones from incubation with OA alone was achieved in the assay at a concentration of 100 μM OA (FIG. 1C). Since fatty acid plasma concentrations are usually above 100 μM in humans on a ketogenic diet OA was assayed at a concentration of 100 μM. Combining 1 μM, 5 μM, or 10 μM C4 with 100 μM OA yielded more PHb than providing 100 μM OA alone (FIG. 2A). Moreover, the PHb production increased with the concentration of C4 provided. The effect of combining C4 and OA on PHb production was more than additive, it cannot be explained by the sum of the individual effects of C4 and OA on PHb production. Therefore, C4 given in a physiological concentration range of 1 μM to 10 μM potentiates the top-level production of ketones from a single fatty acid.

Next, the ketogenic enhancement by C4 of the ketone production from a fatty acid blend contained in a ketogenic diet (K-one) was assessed. This fatty acid blend did not contain C4. Combining 10 μM C4 with 100 μM fatty acid blend produced more βHb than providing the 100 μM fatty acid blend alone (FIG. 2B). The increase in PHb production achieved by combining 10 μM C4 and 100 μM fatty acid blend was more than additive since providing C4 alone yielded no PHb (FIG. 2B).

Liver cells were also co-incubated with a fixed concentration of 10 μM C4 and a concentration range of 1 μM to 100 μM of a fatty acid blend contained in a ketogenic diet (K-one). Control liver cells were exposed to the same concentration range of this fatty acid blend in the absence of C4. C4 increasingly enhanced the production of ketones from the fatty acid blend as the fatty acid blend concentration neared 100 μM (FIG. 2C). These results further demonstrate that a low physiological concentration of C4 potentiates the production of PHb from a fatty acid blend contained in a ketogenic diet. To assess, if C4 may be used as a substrate for ketone synthesis at concentrations of 10 μM and below, a dose-response curve for C4 as a substrate for the production of PHb was obtained, covering a concentration range for C4 from 1 μM to 100 μM. The dose-response curve shows that C4 starts to be turned as a substrate into PHb above a concentration of 10 μM (FIG. 2D). This further corroborates that C4 at concentrations below 10 μM acts as a functional enhancer of the production of ketones from a single fatty acid or a fat blend and not as a direct substrate for ketone synthesis.

To summarize, C4 within a low physiological concentration range of 1 μM to 10 μM potentiates the production of ketones (PHb) from a single fatty acid (oleic acid, OA) (FIG. 2A) and from a blend of fatty acids contained in a ketogenic diet (K-one) (FIG. 2B). The effect of C4 on total ketone production is clearly greater than the sum of the individual contributions of equimolar concentrations of C4 and the fat blend to the total ketone yield. Hence, it is more than additive and therefore not explained by C4 being used as a substrate for ketone production within a concentration range of 1 μM to 10 μM. This is also supported by the dose-response curve for C4, which shows that C4 starts to be effectively turned into ketones at concentrations above 10 μM (FIG. 2D). Therefore, C4 within a low physiological concentration range of 1 μM to 10 μM potentiates the top-level production of ketones from a single fatty acid or a blend of fatty acids.

### 1.3 Effect of C6 on Ketone Production

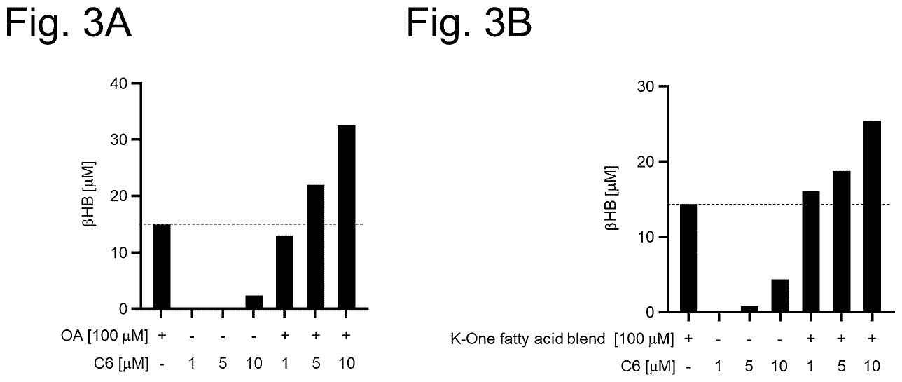

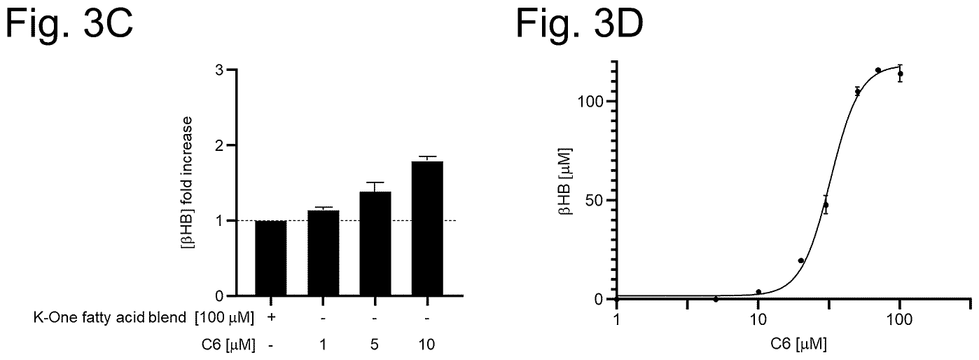

It was found that when performing similar experiments as described in more detail above for C2 and C4 that C6 within a low physiological concentration range of 1 μM to 10 μM potentiates the production of ketones (PHb) from a single fatty acid (oleic acid, OA) (FIG. 3A) and from a blend of fatty acids contained in a ketogenic diet (K-one) (FIGS. 3B and 3C). The effect of C6 on total ketone production from the fat blend was greater than the sum of the individual contributions of equimolar concentrations of C6 and the fat blend to the total ketone yield. Hence, it is more than additive and therefore not explained by C6 being used as a substrate for ketone production within a concentration range of 1 μM to 10 μM. This is also supported by the dose-response curve for C6, which shows that C6 starts to be effectively turned into ketones at concentrations above 10 μM (FIG. 3 D). The data thus indicate that C6 functionally upregulates ketone production within a concentration range of 1 μM to 10 μM. Both, the single fatty acid (oleic acid) and the K-one fat blend were tested at a concentration of 100 μM, when they both maximize ketone production on their own (FIGS. 1C and 1D). The K-one fat blend contains a trace amount of C6 (Table 1). However, the concentration of C6 in a 100 μM K-one blend is 20- to 200-fold below the 1 μM to 10 μM concentration range of C6 at which it enhanced ketogenesis from the blend. Although it is possible that this trace amount of C6 in the K-one fat blend could have already stimulated ketogenesis, our data show that adding C6 within a concentration range of 1 μM to 10 μM further potentiates the top-level production of ketones from a fatty acid blend. In summary, C6 at low physiological concentrations between 1 μM and 10 μM functionally enhances the production of ketones from a single fatty acid or a blend of fatty acids.

### 1.4 Effect of Citrate on Ketone Production

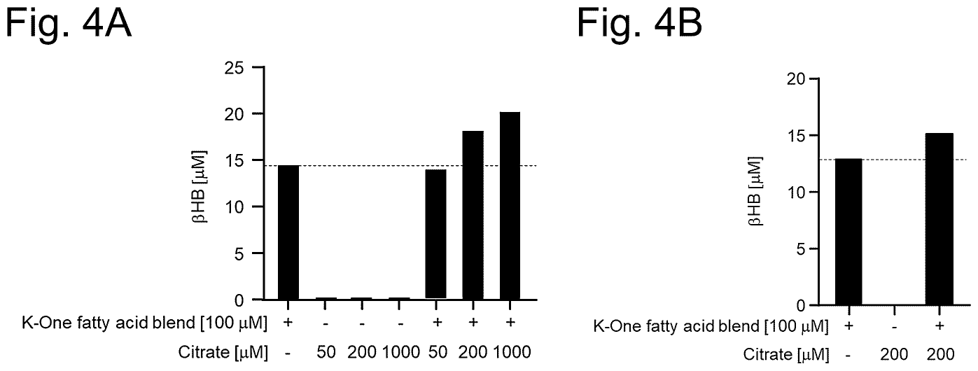

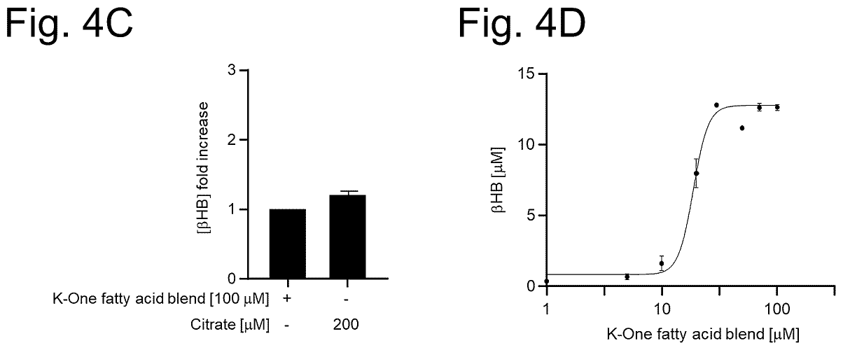

It was found that when performing similar experiments as described in more detail above for C2 and C4 that citrate within a physiological to supraphysiological concentration range of 200 μM to 1000 μM increases the production of ketones (PHb) from a blend of fatty acids contained in a ketogenic diet (K-one) (FIG. 4A-C). The fatty acid blend did not contain citrate (Table 1) and was tested at a concentration of 100 μM, when it achieved top level ketone production on its own (FIG. 1D). The effect of citrate on total ketone production was clearly greater than the sum of the ketone productions achieved individually by the same (equimolar) amounts of citrate or the fat blend (FIGS. 4A and 4B). In fact, providing citrate alone yielded no ketones but when added to the fat blend, it increased the ketone production from the fat blend by ˜10-20% (FIG. 4A-C; FIG. 4C shows the average effect of the experiments shown in FIGS. 4A and 4B for 200 μM citrate, *P<0.05). Hence, citrate potentiates the top-level production of ketones from a blend of fatty acids contained in a ketogenic diet.

### 1.5 Effect of Nicotinamide Riboside on Ketone Production

An assay was developed to measure NAD+ replenishment of NAD+ depleted mouse liver cells (Hepa1-6 cells) by NAD+ precursors under ketogenic diet-like conditions. In this assay, mouse liver cells were starved of glucose and depleted of NAD+ through incubation in a very low glucose (1 mM) medium free of vitamin B3, Trp and FBS and supplemented with 10 nM of an NAD+ depleting drug called FK866. Thereafter, the cells were fed with NR or NAM or Niacin (provided as a combination of NAM and nicotinic acid in a 1:1 molar ratio) or Trp to replenish the cells with NAD+. At the end of the supplementation phase, the cells were scraped and extracted using the NAD+ extraction buffer of the EnzyChrom™ NAD/NADH Assay Kit from BioAssay Systems. The NAD+ content of the cells was determined with the same kit following the manufacturer's manual. The NAD+ measurement is based on a lactate dehydrogenase reaction, in which NAD+ is reduced to NADH, which in turn reduces a formazan (MTT) reagent. The intensity of the reduced product color, measured at 565 nm, is proportional to the NAD+ concentration in the sample. This assay is highly specific for NAD+ and has minimal interference (<1%) by NADP+ or NADPH.

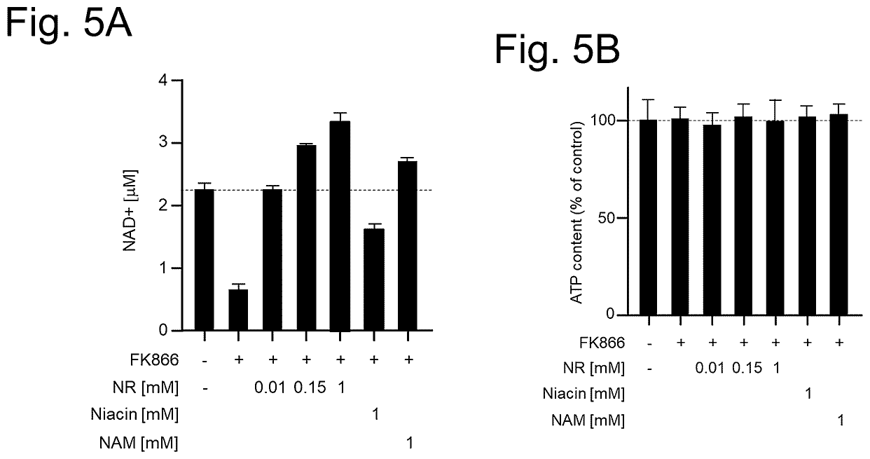

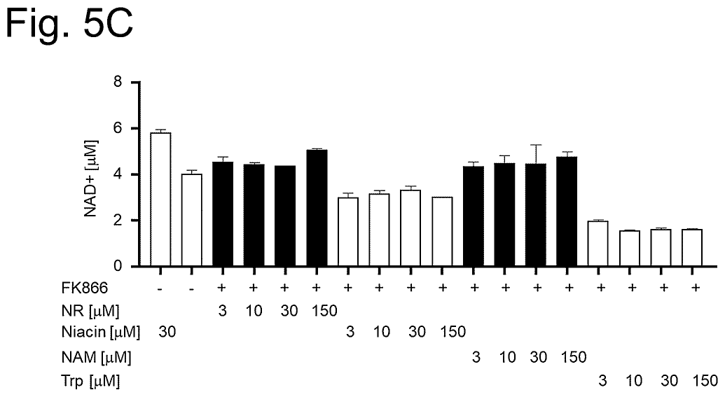

It was found that FK866 treatment significantly depletes liver cells of NAD+ by ˜70% of the baseline NAD+ content of untreated liver cells (FIG. 5A; ****, P<0.0001). Furthermore, it was found that NR provision dose-dependently replenishes NAD+ content to and above this baseline within 24 hours of treatment (FIG. 5A; ****, P<0.0001). Additionally, it was found that NR increases the NAD+ content of NAD+ depleted liver cells to significantly greater extent than equimolar amounts of NAM or Niacin at high concentrations of 1 mM (FIG. 5A; ****. P<0.0001) and to greater extent than equimolar amounts of Niacin at lower concentrations below 150 μM (FIG. 5C). These differences are not due to changes in cell viability as cell counts (determined by ATP content measurements) were equal between different treatments (FIG. 5B). Hence, NR more effectively replenishes NAD+ than NAM and Niacin in NAD+ depleted mouse liver cells.

To measure the impact of replenishing NAD+ in liver cells with NR on ketone production, mouse liver cells were nutrient-deprived in medium containing low (1 mM) glucose and L-carnitine, but no fetal bovine serum, and no NAD+ precursors in the form of vitamin B3 (Niacin, NA, NAM) or Trp, Concomitantly the cells were treated with the NAD+ depleting drug FK866. These conditions created ketogenic diet like nutrient conditions and depleted the cells of NAD+. After 18 hours of incubation, FK866 was removed from the medium and a concentration range of NR was added to replenish NAD+ in liver cells. After 24 hours, the cells were switched to Krebs-Henseleit buffer (KHB) containing L-carnitine and a concentration range (1 μM to 100 μM) of oleic acid, a long chain fatty acid, for 6 hours to induce and measure the production and secretion of b-hydroxybutyrate (PHB) into the medium.

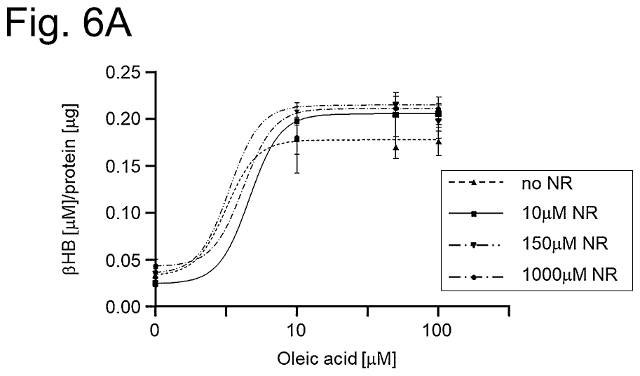

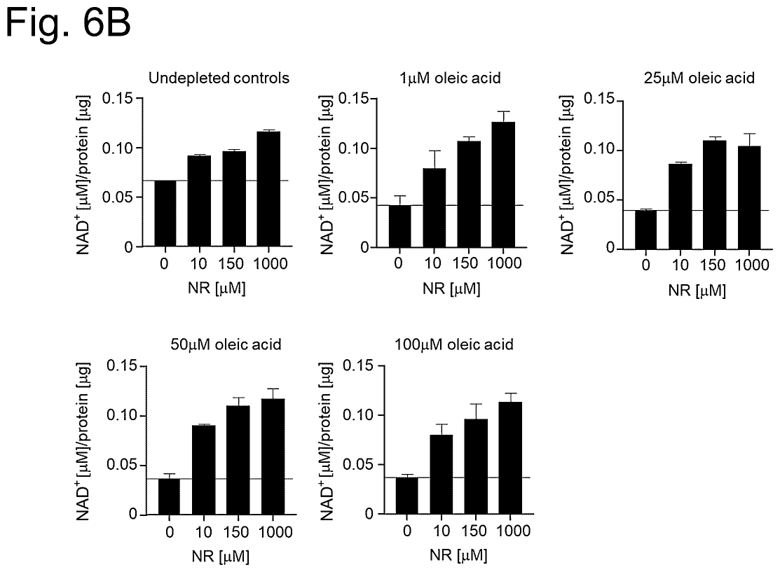

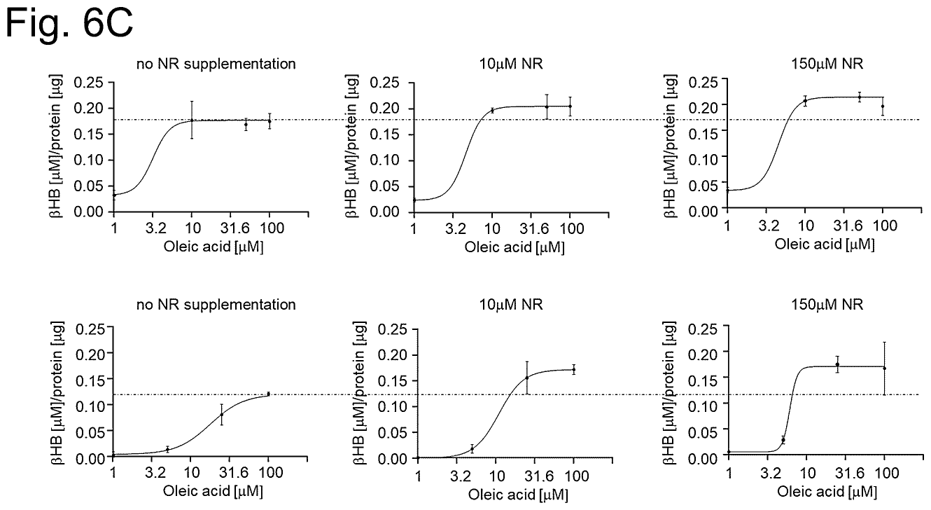

It was found that supplementing NAD+ depleted liver cells with NR at a concentration of 10 μM, 150 μM and 1000 μM increased the top-level production of ketones (FIGS. 6A and 6C) and their NAD+ content (FIG. 6B) under ketogenic diet like nutrient conditions. Combined with a greater ability to replenish NAD+ content (FIG. 5), it is concluded that NR more effectively sustains ketone production than NAM or Niacin or Trp.

### 2 Effect of C3 on Ketone Production

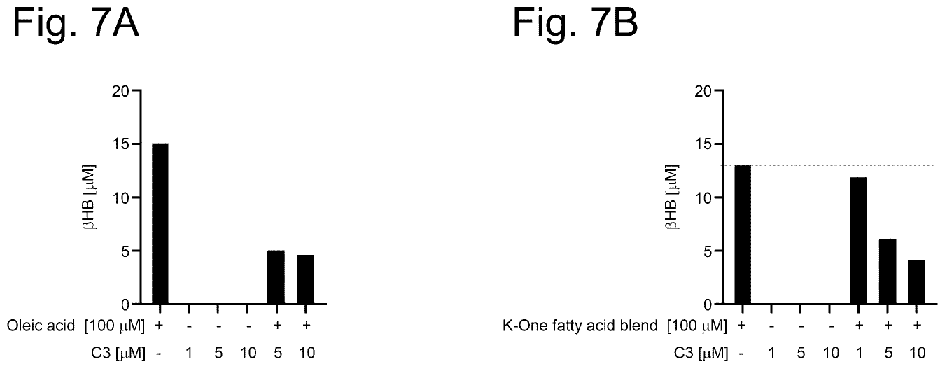

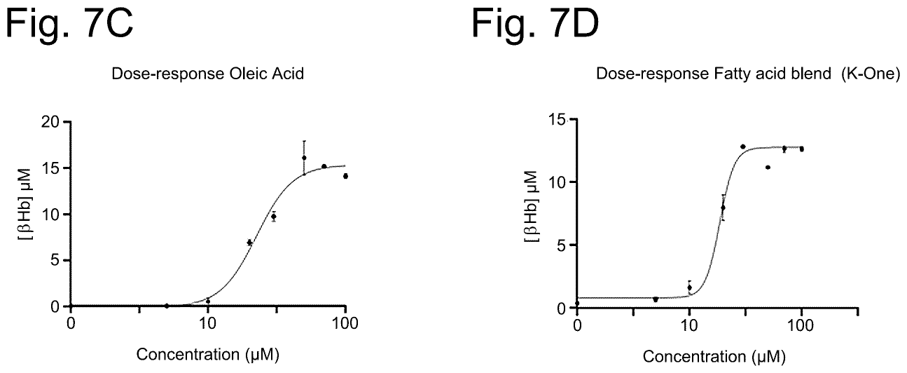

C3 was assayed within a physiological human plasma concentration range from 1 μM to 10 μM. C3 alone at a concentration of 1 μM, 5 μM and 10 μM yielded no PHb (FIGS. 7A and 7B). Assessment of the production of ketones from oleic acid (OA) showed that ketone production was maximized or saturated at a concentration of 100 μM OA (FIG. 7C). The concentrations of individual fatty acid in the blood plasma of humans on a ketogenic diet treatment are usually around or above 100 μM. In combination with the findings illustrated in FIG. 7C it can therefore be assumed that the production of ketones from oleic acid is maximized during a ketogenic diet treatment. Combining 1 μM C3 (data not shown), 5 μM C3, and 10 μM C3 with 100 μM OA yielded less PHb than 100 μM OA alone (FIG. 7A). Hence, C3 provided within a physiological concentration range decreased the top-level production of ketones from OA that would be achieved with a ketogenic diet treatment. Therefore, adding C3 to a ketogenic diet decreases the production of ketones from a single fatty acid.

A blend of medium, long, and very long chain fatty acids contained in a ketogenic diet (denoted as K-one) resulted in top level production of PHb at a total fatty acid concentration of 100 μM (FIG. 7D). The total fatty acid concentration in the blood plasma of humans on a ketogenic diet vastly exceeds 100 μM. It can therefore be assumed that the top-level production of ketones by liver cells from the ketogenic diet's fatty acid blend is achieved during a ketogenic diet treatment. Therefore, the fatty acid blend was tested at a concentration of 100 μM. The blend did not contain C3 (Table 1). Combining 1 μM C3, 5 μM C3, and 10 μM C3 with 100 μM fatty acid blend reduced the PHb yield by more than 60% compared to providing 100 μM fatty acid blend alone (FIG. 7B). Furthermore, C3 decreased the top-level production of βHb from 100 μM fatty acid blend in a dose-dependent manner (FIG. 7B). Therefore, adding C3 to a ketogenic diet decreases the production of ketones from a fatty acid blend contained in a ketogenic diet.

In summary, C3 or propionate within a low physiological concentration of 1 μM to 10 μM inhibits the top-level production of ketones (PHb) from a single fatty acid (oleic acid, OA) (FIG. 7A) and from a blend of fatty acids contained in a ketogenic diet (denoted as K-one) (FIG. 7B). Importantly, the fat blend did not contain C3 (Table 1), meaning that the inhibition is indeed due to the administration of a low concentration of C3. Furthermore, the single fatty acid (oleic acid) and the fat blend (K-one) were tested with C3 at a concentration of 100 μM, when they both maximize ketone production on their own (FIGS. 7C, D) and would maximize the ketone production of humans on a ketogenic diet. Hence, C3 inhibits the top-level production of ketones from a single fatty acid or a blend of fatty acids under ketogenic diet-like treatment conditions.

### Propionate Viability Assay

An assay to measure ketone production in mouse liver cells (Hepa1-6 cell line) was used as described above wherein cells were first grown to a monolayer (growth phase), then exposed to starvation to deplete cellular lipid stores and subsequently brought into a ketogenic phase wherein cells were cultured in presence of oleic acid or a ketogenic diet fat blend with or without the addition of propionic acid (C3). Cells were exposed to C3 during the 6-hour ketogenic phase for assessment of short-term effects of C3 or during the 24-hour starvation plus 6-hour ketogenic phase for assessment of longer-term effects of C3. The impact on cell viability was assessed by measuring the ATP content of the cells as a proxy for the number of alive cells using a bioluminescence assay.

Growth phase: Cells were seeded in 6-well plates at a concentration of 90700 cells/cm2 and allowed to grow for 24 hours at 37° C. in Dulbecco's Modified Eagle Medium (DMEM) supplemented with 5 mM glucose, 10% Fetal Bovine serum (FBS), 4 mM L-glutamine, 1 mM pyruvate, and antibiotics (1% penicillin & streptomycin).

Starvation phase: At the end of the growth phase the cells formed a monolayer. The medium was exchanged for DMEM containing 1 mM glucose and antibiotics (1% penicillin & streptomycin) but no FBS. The medium was further supplemented with L-carnitine (a required co-factor for the uptake of fatty acids into the mitochondria, where fatty acids are turned into ketones). The cells were further supplemented with a concentration range of C3 from 300 uM to 10 mM. The cells were incubated in this medium for 24 hours. The cells in the control conditions were incubated in the same starvation medium without addition of C3.

Ketogenic phase: At the end of the starvation phase, the medium was exchanged for Krebs-Henseleit buffer (KHB) supplemented with the L-carnitine and C3 in a concentration range from 300 uM to 10 mM. The cells in the control conditions were incubated in the same starvation medium without addition of C3. After 6 hours of incubation, the number of alive cells is measured by measuring the ATP content with ATP Determination Kit with recombinant firefly luciferase and its substrate luciferin (provided by ThermoFischer, ATP Determination Kit 200-1,000 assays, catalogue number: A22066).

Calculation of impact on cell viability: The impact on cell viability caused by C3 was determined by calculating the number of alive cells exposed to C3 as a percentage of the number of alive cells in the control condition for each dose of C3 in n=8 replicates. Statistically significant differences in alive cell numbers were assessed by t-test for each dose.

ATP is the energy source of all living cells and is involved in many vital biochemical reactions. When cells die, they stop synthesizing ATP and the existing ATP pool is quickly degraded. Therefore, ATP is widely accepted as a marker of viable cells. Higher ATP concentration indicates higher number of living cells. ATP assays are procedures that can measure cell viability based on detection of ATP. All living cells, including bacteria, can be detected with ATP assays. Several detection methods can be used, such as colorimetric, fluorescent and bioluminescent. In the present invention bioluminescent ATP assays were chosen to measure cell viability due to higher sensitivity, simple and homogeneous protocol, fast and accurate results.

### 2.1 Effect of Propionate on Cell Viability and Cytotoxicity

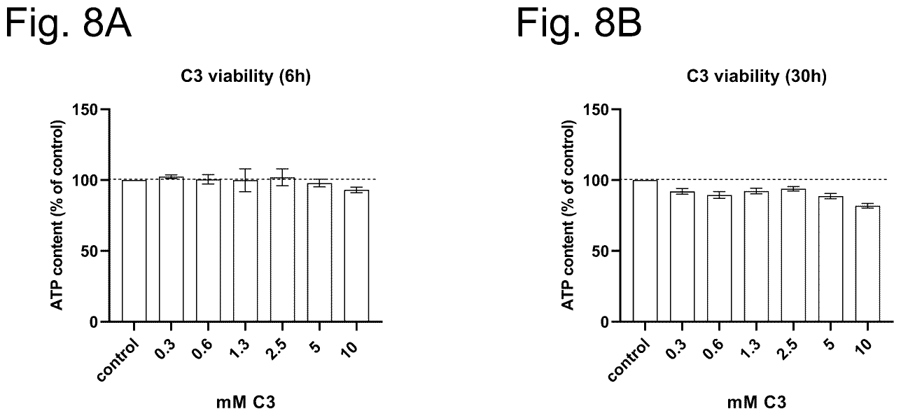

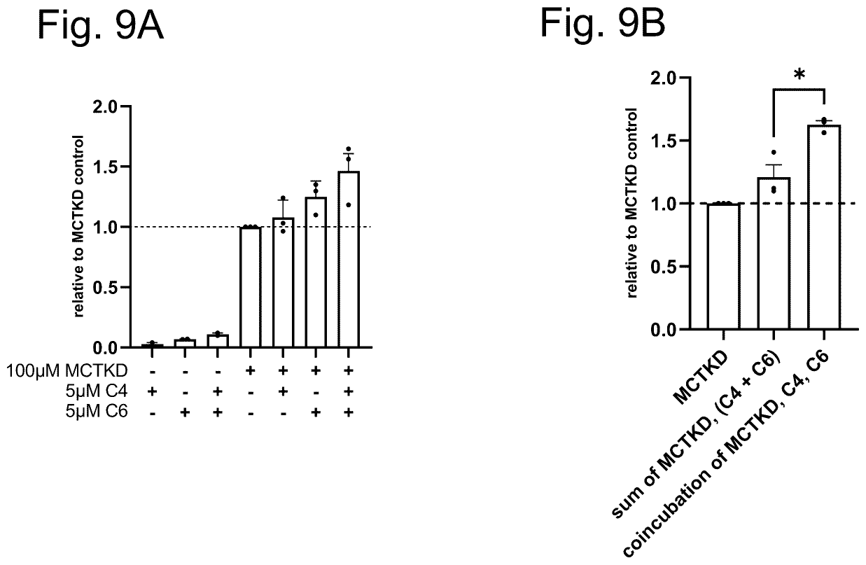

Assessment of the cytotoxic impact of C3 showed that increasing the concentration of C3 in an unphysiological range from 300 mM to 10 mM causes a dose-dependent increase in cytotoxicity. FIGS. 2A and 2B shows that increasing the concentration of C3 decreases the concentration of ATP. When the concentration of ATP decreases, the number of living (and healthy) cells also decreases. Cytotoxicity was already noted for the highest (10 mM) concentration after just 6-hours of exposure to C3 (FIG. 8A) and it became evident for the entire dose-range (from 300 uM to 10 mM) in the long-term assessment (FIG. 8B).

It can therefore be concluded that using low physiological concentration of C3 (1 μM to 10 μM) is necessary to inhibit the top-level production of ketones from a single fatty acid or a blend of fatty acids under ketogenic diet-like treatment conditions without compromising the cell's healthiness and viability.

### 3.1 Effect of the Combination of C4 and C6 on Ketone Production

It was found that when performing similar experiments as described above for the single ingredients with a combination of C4 and C6 at a low physiologic amount of 5 μM each the production of ketones (PHb) from a blend of fatty acids contained in a ketogenic diet (K-one) (FIG. 9A) was enhanced in a more than additive manner. In the absence of a ketogenic fat blend providing C4 or C6 alone or in combination with each other yielded hardly any ketones, but when C4 and C6 were added in combination to the fat blend, it increased the ketone production from the fat blend by ˜40%. The observed increase was to an extent higher than could be predicted on the basis of the effect on ketone production observed for C4 and C6 individually (bars 3 and 4 versus the 6th bar of FIG. 9A respectively). The dotted line represents the threshold ketone production induced by the ketogenic fat blend. FIG. 9B shows the synergy between the ketogenic fat blend and the combination of C4 and C6; the middle bar shows the expected effect on ketone production by adding up the sum of the effect on ketone production observed with C4 and C6 individually, the 3th bar on the right-hand side shows the actual synergistic effect observed of coincubation of C4 and C6. In conclusion C4 and C6 together synergistically boost top-level ketone production from a ketogenic fat blend.

### 3.2 Effect of the Combination of C4, C6, Citrate and NR or NAM on Ketone Production

In a next assay the boosting or potentiating effect of 5 μM C4, 5 μM C6, 200 μM citrate either with 10 μM NR or 10 μM NAM was assessed.

The experimental set-up was similar to the set-up for the single ingredients as described here above with the exception that the medium during the starvation phase was adapted:

At the end of the growth phase when the cells formed a monolayer medium was exchanged for DMEM containing 1 mM glucose and antibiotics (1% penicillin & streptomycin) but no FBS. The medium was further supplemented with L-carnitine (a required co-factor for the uptake of fatty acids into the mitochondria, where fatty acids are turned into ketones). For the specific assessment of the effect of NR or NAM in the combination of ingredients, the medium was additionally deprived of Vitamin B3 and tryptophan since tryptophan can compensate for the lack of B3 as an NAD+ precursor.

At the end of the starvation phase, the medium was exchanged for Krebs-Henseleit buffer (KHB) supplemented with the L-carnitine and the test nutrients used were as follows:
i) full combination of C4, C6, citrate and NR
ii) full combination of C4, C6, citrate and NAM
iii) ketogenic fat blend
iv) full combination of C4, C6, citrate and NR with a ketogenic fat blend
v) full combination of C4, C6, citrate and NAM with a ketogenic fat blend

ßHB production was subsequently assessed as described above. It was found that in the absence of a ketogenic fat blend both the combination with NR as the combination with NAM did not result in a substantial ketone production (FIG. 10). The use of a ketogenic fat blend alone resulted in the production of ketone production, reflected by the dashed line in the graph and herewith denominated the threshold level of ketone production. The further addition of either the combination with NR or the combination with NAM to the ketogenic fat blend resulted in a significant increase in ketone production above this threshold level. Surprisingly, the increase above the threshold level conveyed by either combination was greater than the ketone yields that is achieved individually by either combination in the absence of the ketogenic fat blend. Therefore, this result demonstrates a more than additive that is a synergistic effect of the combination with NR as with the combination with NAM on ketone production from a ketogenic fat blend. Therefore, the combination of C4, C6, citrate and either NR or NAM was found to potentiate top-level ketone production from a ketogenic fat blend.

### 4. Dietary Fiber Fermentation Assessment

A standard adult human gut microbiota pool was used to assess the fermentation of dietary fibers. This pool was established via fecal donations from 6 healthy adult volunteers (Caucasian individuals, age 25-60 years, no antibiotic use in the 3 months preceding the donation, self-assessment of health status). Before starting the fermentation with the test compounds, the standardized fecal adult pool was incubated in SIEM (standard ileal effluent medium) under anaerobic conditions overnight (37° C.; 300 rpm) in orderto activate the bacteria as described in Schuren F et al. The i-screen: A Versatile Preclinical Platform for Gut Microbiota Studies. J Prob Health. 7:212, 2019.

The dietary fibers were tested at a concentration of 6-10 mg/ml, depending on their viscosity and/or fermentability. All fibers were tested in triplicates. The following conditions were applied:
Partially hydrolysed guar gum (Benefibre & Optifibre) 6.6 mg/ml
Inulin (Orafti HP) 10 mg/ml
GOS (Vivinal GOS) 6 mg/ml
Locust bean gum (LBG) 6 mg/ml
Soy fiber (Fibrim) 10 mg/ml
Soy fiber (Fuji) 10 mg/ml
Cellulose (Vitacel) 10 mg/ml
Yeast beta-glucan (YBG) 10 mg/ml
Mix A, containing GOS, inulin, soy fiber (Fuji), resistant starch (Novelose 330) 10 mg/ml
Mix B, containing FOS, inulin, soy fiber (Fibrim), resistant starch (Novelose 330), acacia gum, cellulose (Vitacel) 6.6 mg/ml

In each run a negative control was included (a blank control medium with only the fecal pool) and all conditions started at pH 6.3. After 24 hours of anaerobic fermentation in SIEM medium at 37° C., samples were collected for pH measurements and metabolite analysis.

The pH was measured by immersing a 423 pH-electrode (Mettler Toledo, Columbus, OH, USA), connected to a Handy-lab pH meter (Schott Glas, Mainz, Germany), directly in a sample.

SCFA covering acetate, propionate, and n-butyrate and branched chain fatty acids (BCFA) covering iso-butyrate and iso-valerate were analyzed as described by Jouany et al (1982) with modifications as described by Van Nuenen et al. (2003). Briefly, fermented material from the i-screen samples was centrifuged (˜12,000 g, 5 min). Cells were removed from the supernatant by filter sterilization (0.45 μm). A mixture of formic acid (20%), methanol and 2-ethyl butyric acid (internal standard, 2 mg/ml in methanol) was added. A 3 μl sample with a split ratio of 75.0 was injected on a GC-column (ZB5HT inferno, ID 0.52 mm, film thickness 0.10 μm; Zebron; Phenomenex, USA) in a Shimadzu GC-2014 gas chromatograph.

It was found that after 24 hr of fermentation inulin, GOS, the soy fibers, cellulose, and both mixes result in a higher butyrate/propionate ratio as compared to the blank control (>1.2) (FIG. 11). Dietary fibers and mixtures of such fibers having a butyrate to propionate ratio of at least 1 are considered butyrogenic dietary fibers according to the invention. In contrast the partially hydrolysed guar gums, LBG, and YBG remain below a butyrate/propionate ratio of 1.

### 5. Effect of the First and Second Composition

A non-limiting example of the use of the first and second composition may be as follows: A 70 kg male subject who will start a ketogenic diet will be treated with the first and second composition to control ketosis. The subject will consume about 3000 kcal per day of the ketogenic diet. The blood level of ketones is followed daily using testing strips. Before each meal the subject will consume the first composition. Consequently, the subject consumes three times per day a first composition comprising 7 mg butyric acid and 455 mg caproic acid. When blood ketone levels rise above 3 mmol/L as determined with test sticks the subject will be administered one or more doses of the second composition comprising 6 mg propionic acid per dose.

In a further non-limiting example of the use of the first and second composition may be as follows: A 70 kg male subject who will start a ketogenic diet will be treated with the first and second composition to control ketosis. The subject will consume about 3000 kcal per day of the ketogenic diet. The blood level of ketones is followed daily using testing strips. Before each meal the subject will consume the first composition. Consequently the subject consumes three times per day a first composition comprising 14 mg butyric acid, 910 mg caproic acid, 1.4 g citrate and 84 mg NR and in addition optionally 70 mg acetic acid. When blood ketone levels rise above 3 mmol/L as determined with test sticks the subject will be administered one or more doses of the second composition comprising 12 mg propionic acid per dose.

1. A first composition providing at least one of butyric acid or caproic acid and in addition optionally acetic acid for use in controlling ketosis associated with a ketogenic diet in a subject adhering to a ketogenic diet,
wherein the at least one of butyric acid or caproic acid and in addition optionally acetic acid is comprised in non-ketogenic amounts in the first composition, or the at least at least one of butyric acid or caproic acid and in addition optionally acetic acid is provided in non-ketogenic amounts by a butyrogenic dietary fiber or a butyrogenic dietary fiber blend comprised in the first composition,
wherein the therapeutic use involves
a) administering the first composition to the subject,
c) optionally administering a second composition comprising propionic acid to reduce ketone production/administering a second composition comprising propionic acid provided that blood ketone levels are above 3 mmol/L.

2. The first composition for use according to claim 1, wherein the therapeutic use further involves b) measurement of blood and/or urine ketone levels.

3. The first composition for use according to claim 1, wherein the first and second composition are for sequential use, optionally more than one sequential use.

4. The first composition for use according to claim 1, wherein the subject adhering to a ketogenic diet is suffering from a condition selected from the group of conditions consisting of
i) neurological diseases including epilepsy, stroke, traumatic brain injury, migraine;
ii) neurodegenerative diseases including Alzheimer's disease, Parkinson's disease and age-related mild cognitive impairment (MCI);
iii) neuromuscular diseases including amyotrophic lateral sclerosis, ageing-induced muscle inactivation, muscle wasting and sarcopenia, or elderly at risk of developing or suffering from frailty;
iv) psychiatric diseases including attention deficit hyperactivity disorder (ADHD) and autism spectrum disorders;
v) metabolic conditions including, insulin resistance, type-1 and type-2 diabetes mellitus, Glucose transporter type 1 (Glut1) deficiency, Pyruvate dehydrogenase (PDH) deficiency, glycogen storage diseases and mitochondrial diseases;
vi) oncological disorders;
vii) cardiovascular diseases leading to heart failure.

5. The first composition for use according to claim 1, wherein the first composition further comprises one or more compounds selected from citrate, nicotinamide riboside and nicotinamide.

6. The first composition for use according to claim 1, wherein the first composition further comprises:
(i) butyric acid
(ii) caproic acid
(iii) citrate, and
(iv) nicotinamide riboside or nicotinamide.

7. The first composition for use according to claim 1, wherein the first composition is administered at a dose of
(i) 0.2 mg to 12 mg per kilogram bodyweight per day of butyric acid
(ii) 6 mg to 165 mg per kilogram bodyweight per day of caproic acid
(iii) 0.01 gram to 0.4 gram per kilogram bodyweight per day of citrate, and
(iv) 0.5 mg to 15 mg per kilogram bodyweight per day of nicotinamide riboside.

8. The first composition for use according to claim 1, wherein the first composition enhances ketosis in a subject adhering to a ketogenic diet.

9. The first composition for use according to claim 1, wherein the second composition (i) decreases ketone production and/or
(ii) prevents and/or treats ketoacidosis,
wherein (i) decreasing ketone production and/or (ii) preventing and/or treating ketoacidosis includes lowering blood ketone levels below 3 mM.

10. A propionic acid for therapeutic use in
i) controlling the effect of a ketogenic diet and/or
ii) controlling ketone body production
in a subject adhering to a ketogenic diet and using a first composition providing at least one of butyric acid or caproic acid and optionally acetic acid wherein the at least one of butyric acid or caproic acid and in addition optionally acetic acid is comprised in non-ketogenic amounts in the first composition, or the at least at least one of butyric acid or caproic acid and in addition optionally acetic acid is provided in non-ketogenic amounts by a butyrogenic dietary fiber or a butyrogenic dietary fiber blend comprised in the first composition.

11. The propionic acid for therapeutic use according to claim 10, wherein i) controlling the effect of a ketogenic diet and/or ii) controlling ketone body production involves lowering blood ketone bodies to a range between 1.0 to 3.0 mmol/L.

12. The propionic acid for therapeutic use according to claim 10, wherein the therapeutic use involves
a) administering the first composition,
b) administering a second composition comprising propionic acid to reduce ketone production/administering a second composition comprising propionic acid.

13. The propionic acid for therapeutic use according to claim 12, wherein the therapeutic use further involves c) measurement of blood and/or urine ketone levels.

14. A kit of parts comprising:
1) a ketogenic diet composition having a ketogenic weight ratio between 1:1 and 4:1;
2) a first composition comprising at least one of butyric acid or caproic acid and optionally acetic acid wherein the at least one of butyric acid or caproic acid and in addition optionally acetic acid is comprised in non-ketogenic amounts in the first composition, or the at least at least one of butyric acid or caproic acid and in addition optionally acetic acid is provided in non-ketogenic amounts by a butyrogenic dietary fiber or a butyrogenic dietary fiber blend comprised in the first composition;
3) a second composition comprising propionic acid.

15. (canceled)
